(19) United States
(12) Patent Application Publication
(10) Pub. No.: US 2026/0363778 A1
(43) Pub. Date: Nov. 19, 2026

(54) EVALUATION OF FLUID OR AIR FLOW OBSTRUCTION

(71) Applicant: MATERIALISE NV, Leuven (BE)

(72) Inventors: Polina KARABACH, Leuven (BE); Anton CHEBOTAREV, Leuven (BE); Anneleen VAN ASSCHE, Leuven (BE)

(73) Assignee: MATERIALISE NV, Leuven (BE)

(21) Appl. No.: 19/487,853

(22) PCT Filed: May 31, 2024
(86) PCT No.: PCT/EP2024/065094

§ 371 Date: Nov. 25, 2025

(30) Foreign Application Priority Data
May 31, 2023 (NL) 2034956

(51) Int. Cl. A61B 34/10; G16H 20/40; G16H 50/50

(52) U.S. Cl. CPC A61B 34/10; G16H 20/40; G16H 50/50; A61B 2034/102; A61B 2034/105; A61B 2034/108

(57) ABSTRACT

A computer-implemented method for creating a treatment plan for a medical treatment on an anatomical structure of interest of a patient, said structure comprising a fluid or air passageway defining a fluid or airflow path, said process comprising: displaying a graphical representation of the anatomical structure; adapting the graphical representation according to a treatment plan; determining a region of interest within the anatomical structure; determining a plurality of evaluation planes through the region of interest; determining for each evaluation plane, a cross-sectional size; displaying feedback regarding the treatment plan, wherein said feedback comprises information regarding at least one cross-sectional size corresponding to at least one of the plurality of evaluation planes; and if one or more of the at least one cross-sectional size is below a predetermined value, adapting the treatment plan, adapting the graphical representation according to the adapted treatment plan and updating the displayed feedback in real time.

### BACKGROUND

### Field

This application relates to evaluation of fluid and/or air flow through an anatomical structure in a patient's anatomy. In some aspects, this application relates specifically to determining a degree of obstruction in a fluid or air passageway of the anatomical structure, such as a valve, artery, vein, tract, airway, etc.

### Description of the Related Technology

For many cardiac, cardiovascular, and/or respiratory conditions, the incorporation of prosthetic devices into a patient's anatomy is becoming a common treatment option. However, the presence of such a device in the heart, in a blood vessel, or in a part of the respiratory tract may disrupt, or partially or entirely obstruct, the natural flow of blood or air. For example, in treatments such as transcatheter mitral valve replacement (TMVR), mitral valve-in-valve (ViV), valve-in-ring (ViR), and valve-in-MAC (ViMAC) procedures, obstruction of the left-ventricle outflow tract (LVOT) is a potentially lethal complication.

For example, TMVR, ViV, ViR, and calcific mitral valve procedures can lead to an elongation of the LVOT. This elongation is confined by the deflected anterior mitral valve leaflet, deflected bioprosthetic leaflets, and/or transcatheter heart valve struts. That is, incorporation of a prosthetic device may deflect the anterior mitral valve leaflet and may cause it to remain open, thereby at least partially obstructing the natural flow of blood from the LVOT.

Similarly, in transcatheter aortic valve replacement (TAVR), parts of the prosthetic valve can partially or entirely obstruct blood flow towards the left and/or right coronary arteries, leading to a potentially life-threatening complication.

Pre-procedural evaluations of a patient may be performed to estimate whether and to what degree a prosthetic device may disrupt the natural flow of blood or air in the patient. For example, a pre-procedural evaluation of possible obstructions of blood flow through the LVOT may be performed using a two-dimensional (2D) cardiac computed tomography (CT) image. Using the CT image, measurements can be made at cross sections of the LVOT to determine a smallest cross section of the LVOT area.

However, this procedure presents several shortcomings. For example, each 2D CT image is limited to a single cross section of any anatomical region of concern. For instance, an LVOT area may include irregularities in shape and size that cannot be seen in a particular 2D CT image. Thus, an irregular shape of the LVOT not shown in the 2D CT image reduces the quality of the pre-procedural evaluation. Moreover, if the procedure is performed manually, this leaves room for inter-user and intra-user variability and error. For example, due to the complex three-dimensional shape of the heart, manually determining the LVOT and the extent of a possible obstruction of the LVOT is not a straightforward task.

### SUMMARY

Certain aspects relate to a method for creating a treatment plan for a medical treatment on an anatomical structure of interest of a patient, said anatomical structure of interest comprising a fluid or air passageway defining a fluid or airflow path. The method may include displaying a graphical representation of at least part of the anatomical structure of interest. The method may also include adapting the graphical representation of the anatomical structure of interest according to a treatment plan. The method may further include determining a region of interest within the anatomical structure of interest. The method may further include determining a plurality of evaluation planes through the region of interest. The method may further include determining for each evaluation plane of the plurality of evaluation planes a cross-sectional size for said evaluation plane. The method may further include displaying feedback regarding the treatment plan, wherein said feedback comprises information regarding at least one cross-sectional size corresponding to at least one of the plurality of evaluation planes. The method may further include if one or more of the at least one cross-sectional size is below a predetermined value, adapting the treatment plan and updating the displayed feedback in real time.

Certain aspects relate to an apparatus for creating a treatment plan for a medical treatment on an anatomical structure of interest of a patient, said anatomical structure of interest comprising a fluid or air passageway defining a fluid or airflow path. The apparatus comprises a memory and a processor communicatively coupled to the memory. The processor and the memory may be configured to perform a method for creating a treatment plan for a medical treatment on an anatomical structure of interest of a patient, said anatomical structure of interest comprising a fluid or air passageway defining a fluid or airflow path.

Certain aspects relate to a non-transitory computer-readable storage medium comprising instructions that, when executed by a processor of an apparatus cause, the apparatus to perform a method for creating a treatment plan for a medical treatment on an anatomical structure of interest of a patient, said anatomical structure of interest comprising a fluid or air passageway defining a fluid or airflow path.

### BRIEF DESCRIPTION OF THE DRAWINGS

FIGS. 1 and 2 are diagrams illustrating a cutaway view of a heart.

FIG. 3 is an example of a computer environment suitable for implementing certain embodiments described herein.

FIG. 4 is an example of a computing device suitable for implementing various embodiments is shown.

FIG. 5 is an example two-dimensional (2D) computed tomography (CT) image of a patient's heart.

FIG. 6 is an example of a combination of multiple orthogonal 2D images that are used to generate a three-dimensional (3D) model of the patient's heart.

FIG. 7 is a sectional view of a structure of interest from the 3D model of FIG. 6.

FIG. 8 is a sectional view of a structure of interest from the 3D model of FIG. 6.

FIG. 9 is a sectional view of a structure of interest from the 3D model of FIG. 6.

FIG. 10 is a sectional view of an example 2D CT image of a patient's heart.

FIG. 11 is a sectional view of a structure of interest from the 3D model of FIG. 6.

FIG. 12 is a diagram illustrating a conceptual guide curve having a plurality of planes defined orthogonally to the guide curve.

FIG. 13 is a diagram illustrating a conceptual guide curve having a first plane of the plurality of planes.

FIG. 14 is a diagram illustrating the conceptual guide curve having a rotation of the first plane of the plurality of planes.

FIGS. 15 and 16 are example graphs illustrating a cross-sectional area along a centerline or guide curve of a fluid or air passageway.

FIG. 17 is a flow chart showing a process for creating a plurality of section planes, according to certain embodiments.

FIG. 18A and FIG. 18B are illustrations of a user interface of a computer environment suitable for implementing certain embodiments described herein.

FIG. 19 is a flow chart showing a process for creating a treatment plan for a medical treatment according to certain embodiments.

FIG. 20 is an illustration of a user interface of a computer environment suitable for implementing certain embodiments described herein.

### DETAILED DESCRIPTION OF CERTAIN INVENTIVE EMBODIMENTS

The methods and systems described herein are illustrated in the context of the placement of a prosthetic device in the mitral valve of a patient. However, the person skilled in the art will readily appreciate that the same principles may be applied to any treatment to any part of a patient's anatomy that may cause obstruction of blood and/or air pathways (i.e., the structure of interest), such as to other treatments of the mitral valve, to the placement of prosthetic devices in other valves of the heart, such as the aortic valve, the pulmonary valve or the tricuspid valve, to other treatments of these types of valve, to the placement of prosthetic devices in other parts of the patient's anatomy having a blood and/or air pathway, or to other treatments of such parts of the patient's anatomy.

Unless explicitly mentioned, all method steps disclosed herein may be performed manually by a user of a computing device, automatically by a computing device, or semi-automatically by a computing device with some input from a user. The user may include a medical professional or a non-medical professional, such as an engineer or a technician, or any other suitable user.

As noted above, incorporation of prosthetic devices into a patient's anatomy may cause obstruction of blood and/or air pathways. In one example, a prosthetic mitral valve may cause obstruction of the left-ventricle outflow tract (LVOT), thereby reducing blood flow leaving the heart towards the aorta. Here, a prosthetic mitral valve such as a transcatheter mitral valve replacement (TMVR) may change the size and shape of the original LVOT to a modified LVOT. For example, the LVOT may be extended into the left ventricle (the extension referred to herein as a “neo-LVOT”). The neo-LVOT may have a reduced cross section as compared to the original LVOT due to a protrusion of the prosthetic mitral valve into the LVOT.

The amount of fluid (e.g., blood) or air flow through a volume (e.g., valve, vein, artery, LVOT, neo-LVOT, airway, etc.) may be directly related to the minimum cross-sectional area through which the fluid or air flows in the volume as the minimum cross-sectional area may act as the bottleneck for flow through the volume. Accordingly, blood flow through the modified LVOT may be directly related to the minimum cross-sectional area of the neo-LVOT.

Adequate blood flow through the modified LVOT is critical to ensuring patient viability after insertion of the prosthetic mitral valve. Without adequate blood flow, the patient could have complications, which may even lead to death. Accordingly, robust pre-procedural evaluation of a neo-LVOT area can help reduce the chance of complications in a mitral valve replacement by helping to indicate the blood flow through the neo-LVOT prior to the actual mitral valve replacement. It should be noted that although many of the examples discussed herein relate to pre-procedural evaluations for replacing a patient's mitral valve with a prosthetic device, the techniques described herein are also applicable to pre-procedural evaluations performed prior to any other suitable procedures such as other cardiac, cardiovascular or pulmonary procedures, in which the flow of a fluid (e.g., blood), air, or another gas or liquid may be partially or entirely obstructed. Moreover, the techniques disclosed may apply with equal force to evaluation of the effect of non-medically introduced phenomena on fluid and air flow, such as stenoses, blood clots, tumors, etc.

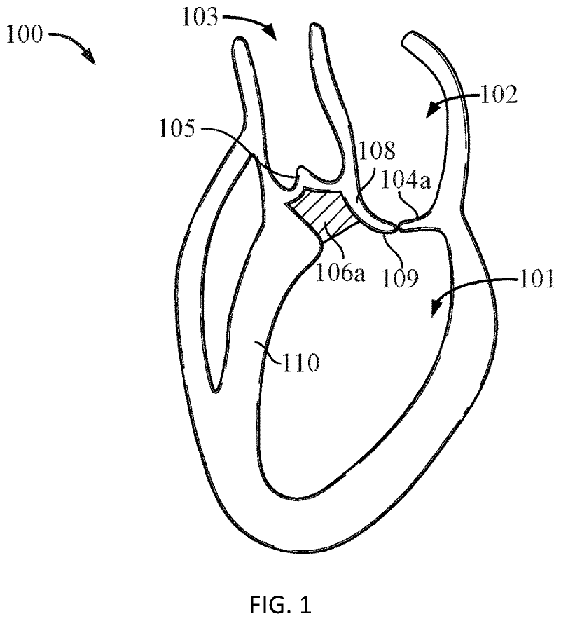

FIG. 1 is a diagram illustrating a sectional view of an example heart structure 100. In particular, the aorta 103, left ventricle 101, and left atrium 102 of heart 100 are shown. The heart structure further includes an aortic valve 105, a left ventricle outflow tract (LVOT) 106a, an intervalvular fibrosa 108, a mitral valve 104a, an anterior mitral valve leaflet (AML) 109, and an interventricular septum (IVS) 110. FIG. 2 is a diagram illustrating a sectional view of another example heart structure 150. In this example, the heart structure 150 includes a prosthetic mitral valve 104b. The placement of the mitral valve 104b in the heart 150 deflects AML 109. The deflected AML 109, along with the anatomy of the IVS 110, defines a neo-LVOT 106b. Optionally, another neo-LVOT region 106c may be added if a user determines that additional measurements are required.

In some methods, the neo-LVOT area is visually estimated by a user by first placing a schematic representation of prosthetic valve 104b in one or more 2D images, and by drawing a 2D curve along an estimate of a centerline of the modified LVOT (i.e., the combination of the original LVOT 106a, the neo-LVOT 106b, and optionally, another neo-LVOT region 106c) on one or more 2D images. The sidewall of prosthetic valve 104b is often assumed to represent the shape of the deflected AML 109. The drawing of this pseudo-centerline may be done manually by a user, and can be prone to error due to the inaccurate visual estimation. The user may then draw a plane that is perpendicular to the pseudo-centerline. The user may select the position of the plane manually at what visually appears to be a small cross section. A distance from the intervalvular fibrosa 108, deflected AML 109 or side wall of prosthetic valve 104b to the wall of the IVS 110 along the plane is then calculated and used as an estimate of the minimum neo-LVOT area. Thus, such an evaluation of the neo-LVOT 106b is crude and may not result in an accurate determination of the minimum neo-LVOT area. For example, multiple visual estimates of the minimum neo-LVOT area for the same heart 100 with the same prosthetic mitral valve 104b placed in the same position May widely vary depending on the user making the estimation and on the choice of 2D image on which the estimation is based. Inaccurate estimations may lead to improper selection and placement of a prosthetic mitral valve 104b in a patient's heart, which could lead to complications or even death.

Unlike such methods, certain embodiments of the systems and methods described herein provide robust and accurate determinations of a degree of obstruction in a fluid or air passageway of an anatomical structure of a patient. That is, certain embodiments of the systems and methods described herein improve the technological field of medical science and medical technology by efficiently and accurately calculating a minimum cross-sectional area of an anatomical region so that a proper prosthetic device can be selected and placed in the patient's anatomy such that the prosthetic device does not obstruct or reduce proper blood and/or air flow through the anatomical region.

In one example, calculating the minimum cross-sectional area of the anatomical region may include calculating one or more of an LVOT 106a and a neo-LVOT 106b/106c. Based on these calculations, a proper prosthetic mitral valve 104b can be selected and placed in a patient's anatomy while maintaining proper blood flow through the neo-LVOT 106b/106c. Such techniques improve the technological field of medical science and medical technology by reducing the chance of patient complication due to improper neo-LVOT 106b/106c calculation and prosthetic mitral valve 104b placement and selection/design. Such techniques further improve the functioning of the computing device itself that is used to calculate the minimum neo-LVOT area 106b/106c by providing an efficient and defined computing system that efficiently finds a minimum cross-sectional area in a volume.

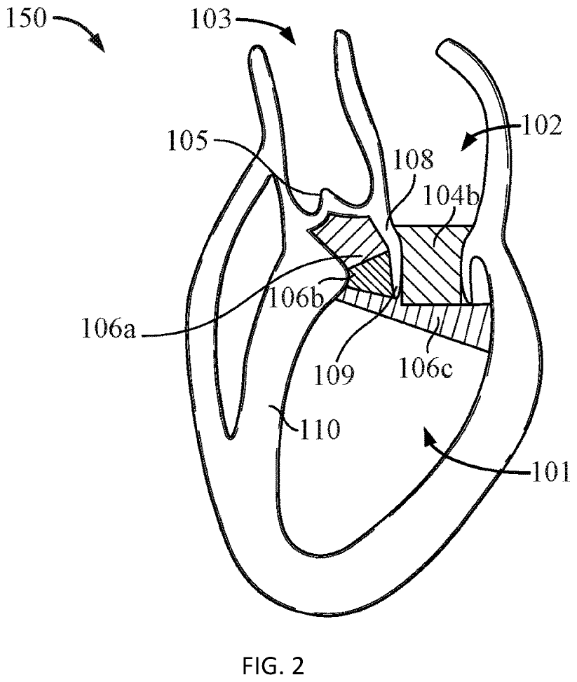

The systems and methods described herein may be implemented in a computing environment comprising one or more computing devices configured to provide various functionalities. FIG. 3 is an example of a computer environment 300 suitable for implementing certain embodiments described herein. The computer environment 300 may include a network 302. The network 302 may take various forms. For example, the network 302 may be a local area network (LAN) installed at a surgical site. In some embodiments, the network 302 may be a wide area network (WAN) such as the Internet. In other embodiments, the network 302 may be a combination of LANs and WANs. Typically, network 302 will allow for secured communications and data to be shared between various computing devices. Among these computing devices is a client device 304. The client device 304 may be a typical personal computer device that runs an off-the-shelf operating system such as Windows, Mac OS, Linux, Chrome OS, or some other operating system. Client device 304 may have application software installed to allow it to interact via the network 302 with other software stored on various other modules and devices in the computing environment 300. This application software may take the form of a web browser capable of accessing a remote application service. Alternatively, the application software may be a client application installed in the operating system of the client device 304. Client device 304 may also take the form of a specialized computer, specifically designed for medical imaging work (e.g., a specialized computer integrated in a medical-imaging appliance or the control system of a medical-imaging appliance), or even more specifically for neo-LVOT area 106b/106c determination. The client device 304 may further take the form of a mobile device or tablet computer configured to communicate via the network 302 and further configured to run one or more software modules to allow a user to perform various methods described herein.

The computer environment 300 may further include image data storage 306. Typically, image data storage 306 takes the form of a large database designed to store image files captured by a scanning device 322. These images may be part of a digital imaging and communications in medicine (DICOM) management system, and may include 2D images (e.g., CT images), 3D images (e.g., images constructed from multiple views in 2D images), and/or 4D images. The image data storage 306 may be part of a scanning device 322, or alternatively it may be part of a client-computing device 304. The image data storage 306 may also be in a standalone database, for example in a server-based system, such as a picture archiving and communication system (PACS), having dedicated storage optimized for medical image data. The computer environment 300 may also include a scanning device 322. The scanning device 322 may typically be a medical imaging device that scans a patient to create images of their anatomy. In computing environment 300 shown in FIG. 3, the scanning device 322 may be a CT scanner, a magnetic resonance imaging (MRI) device or an ultrasound device. However, a skilled artisan will appreciate that other suitable scanning technologies may be implemented which provide imaging data that can be used to create three-dimensional anatomical models.

As will be explained in detail below, the scanning device 322 may be configured to create cross-sectional images of a fluid or air passageway (e.g., anatomy), such as a patient's heart or airway, or a part thereof. Those images may be stored in image data storage 306 and may be utilized to create three-dimensional models of the fluid or air passageway. To that end, computing environment 300 may also include an image processing module 308. The image processing module 308 may take the form of computer software, hardware, or a combination of both which retrieves the medical imaging data from image data storage 306 and generates a three-dimensional model, such as a 3D surface model (e.g., a 3D triangle model), using stacks of 2D image data. The image processing module 308 may be a commercially available image processing software for three-dimensional design and modeling such as the Mimics application from Materialise NV. However, other image processing software may be used. In some embodiments, the image processing module 308 may be provided via a web-based network application that is accessed by a computer over the network (such as client device 304, for example). Alternatively, the image processing module 308 may be a software application that is installed directly on the client device 304, and accesses image data storage 306 via the network 302. In general, the image processing module 308 may be any combination of software and/or hardware located within computing environment 300 which provides image processing capabilities on the image data stored within image data storage 306. The three-dimensional model may be a virtual—or digital—three-dimensional model.

The computing environment also may include a 3D measurement and analysis module 320. The 3D measurement and analysis module 320 may be software that is complementary to and/or bundled with the image processing module 308. In some embodiments, 3D measurement and analysis module 320 and image processing module 308 may be combined into a single module. The 3D measurement and analysis module 320 may include an application configured to determine/estimate a minimum cross section of a fluid passageway, such as neo-LVOT 106b/106c. As will be explained in further detail below, the 3D measurement and analysis module 320 may be generally used to determine precise measurements of various aspects of the fluid or air passageway (e.g., patient anatomy) and a simulated positioning of a feature in the passageway (e.g., obstruction, narrowing, implant such as a prosthetic mitral valve 104b, etc.) in order to determine the minimum cross section of the fluid or air passageway, such as the neo-LVOT 106b/106c. As with the image processing module 308, the 3D measurement and analysis module 320 may be a network-based application which is accessed via a web browser by one or more client devices 304. It may also be a native application installed into the operating system of a computer, such as client device 304 for example. In still other embodiments, the 3D measurement and analysis module 320 may be a network application which is run as a client/server implementation. In certain embodiments, 3D measurement and analysis module 320 may operate on the 3D model generated by image processing module 308. Alternatively or additionally, 3D measurement and analysis module 320 may operate on image data, such as from image data storage 306. Performing measurements on image data, in certain embodiments, makes it possible to eliminate the step of generating a three-dimensional model. However, performing measurements on a 3D model may produce more accurate results, as features such as centerlines and cross sections of anatomical structures may be more accurately determined and detrimental effects of noise or other artefacts in the image data may be reduced. In some embodiments, computer environment 300, image processing module 308, 3D measurement and analysis module 320 and/or the single module combining the functionalities of both image processing module 308 and 3D measurement and analysis module 320 may comprise a user interface 1800 as will be further detailed below.

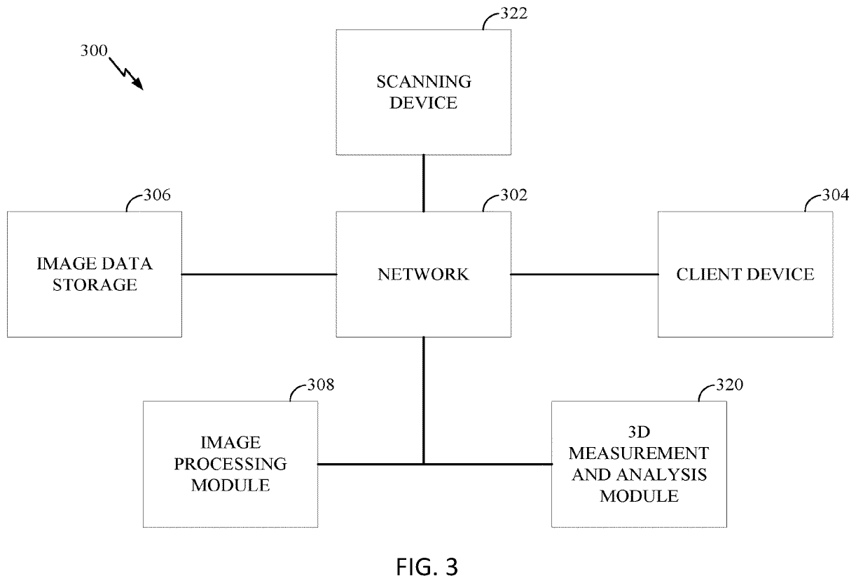

Various embodiments may be implemented using general and/or special-purpose computing devices. Turning now to FIG. 4, an example of a computing device 400 suitable for implementing various embodiments is shown. The computer system 400 may generally take the form of computer hardware configured to execute certain processes and instructions in accordance with various aspects of one or more embodiments described herein. The computer hardware may be a single computer, or it may be multiple computers configured to work together. The computing device 400 includes a processor 403. The processor 403 may be one or more standard personal computer processors such as those designed and/or distributed by Intel, Advanced Micro Devices, Apple, or ARM. The processor 403 may also be a more specialized processor designed specifically for image processing and/or analysis. The computing device 400 may also include a display 404. The display 404 may be a standard computer monitor such as an LCD monitor as is well known. The display 404 may also take the form of a display integrated into the body of the computing device, for example as with an all-in-one computing device or a tablet computer.

Computing device 400 may also include one or more input/output devices 406. These may include standard peripherals such as keyboards, mice, printers, and other basic I/O software and hardware. These may also include display devices, such as computer screens, standalone monitors, or head-mounted displays. The computing device 400 may further include memory 408. Memory 408 may take various forms. For example, memory 408 may include volatile memory 410. The volatile memory 410 may be some form of random-access memory, and may be generally configured to load executable software modules into memory so that the software modules may be executed by processor 403 in a manner well known in the art. The software modules may be stored in a non-volatile memory 413. The non-volatile memory 413 may take the form of a hard disk drive, a flash memory, a solid-state hard drive or some other form of non-volatile memory. The non-volatile memory 413 may also be used to store non-executable data, such database files and the like. In some examples, computing device 400 may include non-transitory computer readable media having code stored thereon for performing the techniques and methods described herein.

Computing device 400 also may include a network interface 414. The network interface may take the form of a network interface card and its corresponding software drivers and/or firmware configured to provide computing device 400 with access to a network (such as the Internet, for example). Network interface 414 may be configured to access various different types of networks, such as those described above in connection with FIG. 3. For example, network interface 414 may be configured to access private networks that are not publicly accessible. Network interface 414 may also be configured to access wireless networks such using wireless data transfer technologies such as EVDO, WiMax, or LTE network. Although a single network interface 414 is shown in FIG. 4, multiple network interfaces 414 may be present in order to access different types of networks. In addition, a single network interface 414 may be configured to allow access to multiple different types of networks.

In general, computing environment 300 shown in FIG. 3 may generally include one, a few, or many different types of computing devices 400 which work together to carry out various embodiments described below. For example, image data storage 306 may be part of a server-based system, such as a PACS system, and may be accessible to the image processing module 308 and/or the 3D measurement and analysis module 320 through network interface 414. A skilled artisan will readily appreciate that various different types of computing devices and network configurations may be implemented to carry out the inventive systems and methods disclosed herein.

### Data Acquisition

In certain embodiments, a user, such as a clinician, engineer, technician, etc., may use a computing device to acquire data. The data may include one or more images of the area of the fluid or air passageway (e.g., anatomy of the patient) to be evaluated. The images may include any suitable (e.g., medical) images for viewing the fluid or air passageway (e.g., computed tomography (CT) scans, etc.). The images may be contrast-enhanced images, such as angiographic images. In some examples, the images may be two-dimensional (2D), three-dimensional (3D), or four-dimensional (4D) images. The 4D images may cover a particular duration of time, such as an entire or partial heart cycle. The 2D or 3D images may include a momentary snapshot of the fluid or air passageway (e.g., end-systole anatomy of the heart structure). In some examples, images may be acquired using scanning device 322. In other examples, images may be acquired by loading images created previously from image data storage 306.

In some examples, computing device 400 may be used to display the images such that a structure of interest, such as in the patient's anatomy, can be visualized. For example, in the case of TMVR, the structure of interest may be a heart, it may be a left ventricle (e.g., the left ventricle 101 of FIG. 1) of a heart, possibly including the mitral valve and the aortic valve (e.g., the mitral valve 104a and the aortic valve 105 of FIG. 1), or it may be the left side of a heart. In some examples, computing device 400 may be used to create a virtual 3D model of the structure of interest. For example, based on the CT images, a virtual 3D model, such as a 3D surface model may be created of the blood pool volume of the heart or a portion thereof (e.g., the left side of the heart or the left ventricle), of any calcifications, of another portion of the anatomy, and/or of any prosthetic devices already present in the patient. Such a 3D (surface) model may be created by segmenting (e.g., medical) images, such as CT images, e.g., by means of image processing module 308. The visualization of the structure of interest may include displaying said 3D (surface) model. In other examples, the visualization of the structure of interest may include one or more 2D (e.g., medical) images, multi-planar reconstruction (MPR) images, or volume-rendered (e.g., medical) images. In some examples, the visualization of the structure of interest may include a display of any combination of 2D, 3D, or 4D images. In some examples, the visualization of the structure of interest may be shown within user interface 1800.

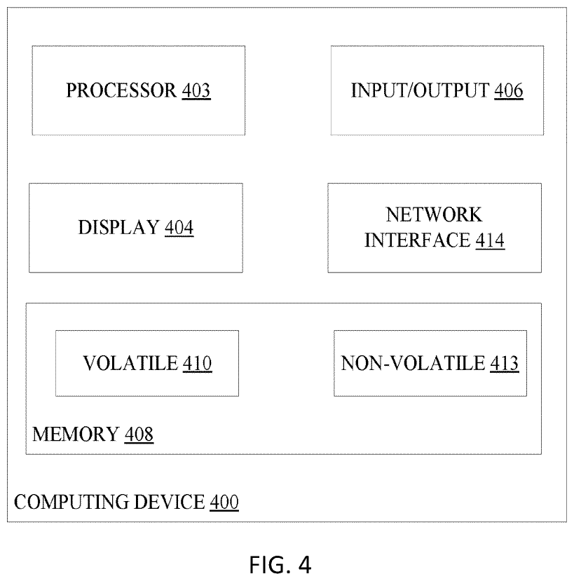

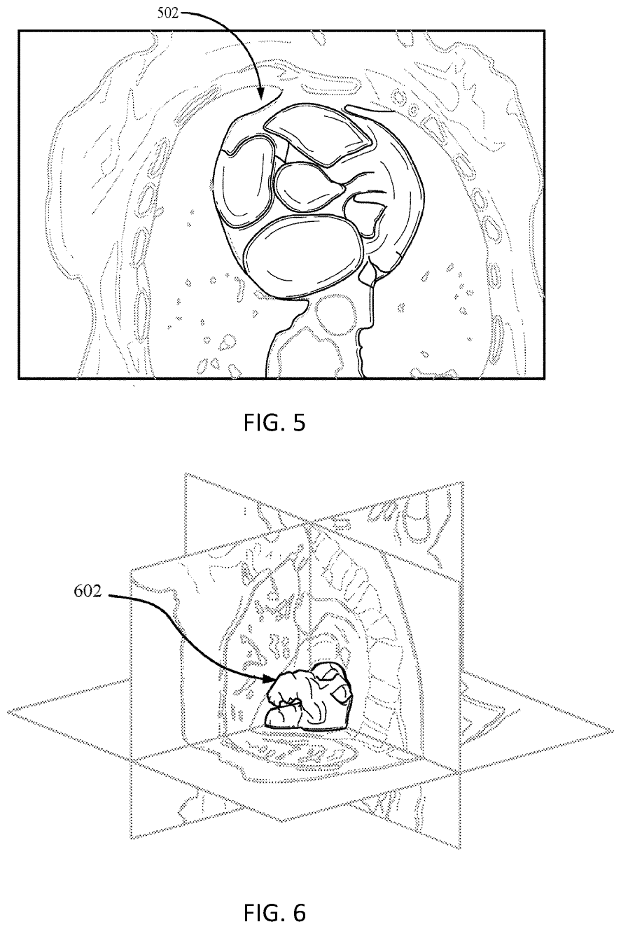

FIG. 5 is an example 2D CT image of a patient's heart 502. As noted, multiple 2D images may be captured of the patient's heart 502 from different perspectives or at different locations, e.g., in order to generate a 3D model of the heart 502 or a portion thereof. For example, FIG. 6 is an example of a combination of multiple orthogonal 2D images that are used to generate a (virtual) 3D model 602 of a structure of interest, such as a 3D surface model, such as the patient's heart 502 or part of the patient's heart. In this example, the image processing module of FIG. 3 may be used to fetch the images from image data storage 306 and combine the 2D images and generate the 3D model 602. In one example, the structure of interest includes the left ventricle 101, the mitral valve 104a, and the aortic valve 105. As such, the 3D model 602 includes a model of these elements of the patient's heart 502 or parts thereof.

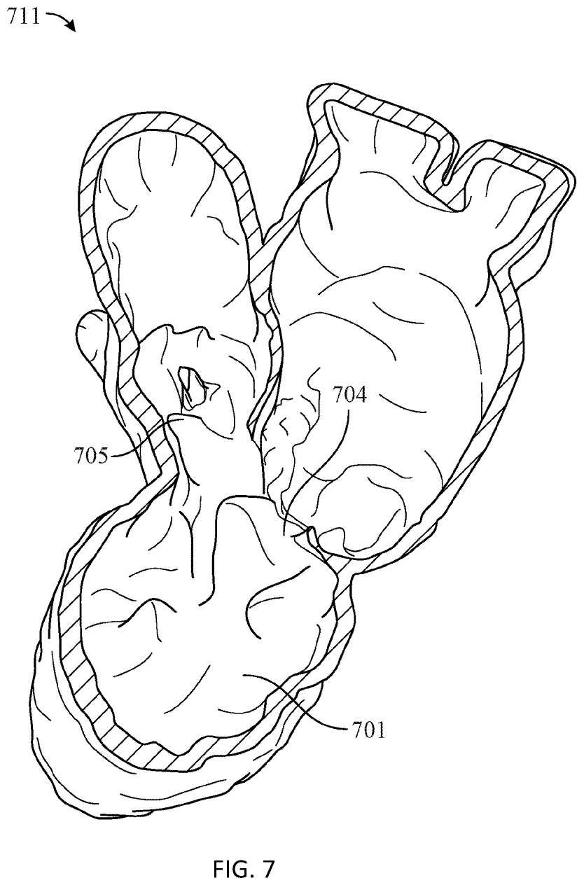

FIG. 7 is a sectional view of a structure of interest 711 from the 3D model 602 of FIG. 6. In some examples, once the 3D model 602 is created, the image processing module 308 may be utilized to generate one or more sectional views of the structure of interest. In this example, the image processing module 308 uses clipping along a plane perpendicular to the mitral valve 704 and the aortic valve 705 so that a user can view the inside of the left ventricle 701. Other clipping planes may be selected. In some examples, the display of the structure of interest 711 may be adapted to reflect a planned post-treatment condition. That is, a user may adapt a graphic representation of the structure of interest 711 to include a virtual model of an implant, such as a prosthetic device. In other embodiments, the adaptation may be performed automatically by computing device 400. For example, using pre-programmed planning algorithms, computing device 400 may identify certain anatomical landmarks, perform certain measurements, automatically select an implant, such as a prosthetic device, and a location for the implant within the structure of interest, and adapt the graphic representation of the structure of interest 711 to include a virtual model of the implant. Semi-automatic approaches, combining automatic algorithms with a certain amount of user input are also possible.

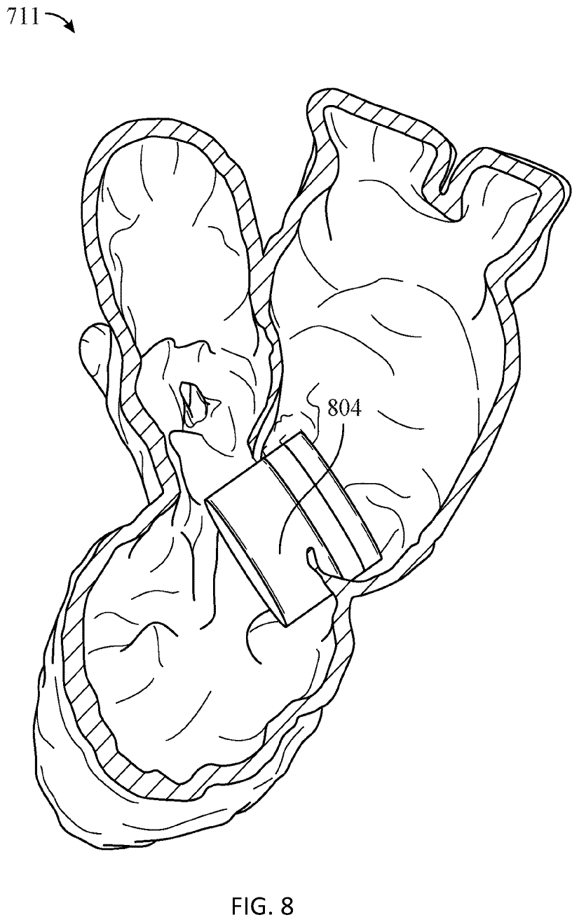

FIG. 8 is a sectional view of a structure of interest 711 from the 3D model 602 of FIG. 6. In this example, the graphic representation of the structure of interest 711 has been adapted to include a virtual model of a prosthetic device 804 (in this example, a prosthetic mitral valve). In some examples, the virtual model of the prosthetic device 804 may be shaped based on its brand, type, and size. In some examples, the adaptation of the graphic representation of the structure of interest 711 may be made based on a planned orientation and location of the prosthetic device 804 relative to the structure of interest. The virtual model of the prosthetic device 804 may be a 2D or 3D model of the prosthetic device 804. It is incorporated into one or more 2D medical images and/or a virtual 3D model of the structure of interest. The virtual model of the prosthetic device 804 may include a schematic representation, such as a cylinder, a truncated cone, a spindle, a double truncated cone or a combination of one or more such or other primitive shapes, and/or a more lifelike 3D model of the actual device. Additionally or alternatively, it may be or include a parameterized virtual object, a model loaded from a file, or a model loaded from a library of device templates. It may be or include a model based on a CAD design of the prosthetic device, on a (e.g., optical) scan of a physical device or on a segmentation of one or more (e.g., medical) images comprising a depiction of a prosthetic device.

Prosthetic device 804 may be virtually placed by means of image processing module 308 or 3D measurement and analysis module 320. The virtual placement of prosthetic device 804 within the patient's anatomy may correspond to the planned outcome of the treatment. For example, a treatment outcome may be planned prior to data acquisition, and may be used to verify legitimacy of the treatment plan through virtual placement of the prosthetic device 804 within the patient's anatomy. In some embodiments, the virtual treatment plan may be created or modified during visualization of the structure of interest 711. For example, the treatment plan may be created or modified by a user, such as a medical professional, or a non-medical professional, such as a technician or engineer, by visually or automatically identifying anatomical structures in the display, and/or based on a planning algorithm dictating prosthetic device 804 selection, location, rotation and/or position. In some examples, the treatment plan may be created automatically, e.g., by computing device 400, based on automatically identified anatomical structures and/or anatomical structures previously identified by a user, such as a medical professional, or a non-medical professional, such as a technician or engineer, and/or based on a planning algorithm dictating prosthetic device 804 selection and location. In some examples, the device size selection and/or the selection of the position (e.g., location and/or orientation) of the device is made with respect to the patient's anatomy. For example, with respect to features of the patient's anatomy, to anatomical landmarks, to geometrical primitives, such as coordinate systems, planes, lines, vectors or points, derived from such features or landmarks, or to measurements taken of or among such features, landmarks or geometrical primitives. In other embodiments, the acquisition of a virtual treatment plan may comprise loading a virtual treatment plan made earlier from a file, a database or any other medium. Any suitable landmark-detection techniques known in the art, such as deep-neural-network methods, may be employed to automatically detect anatomical landmarks.

Determination of a Region of Interest within the Structure of Interest

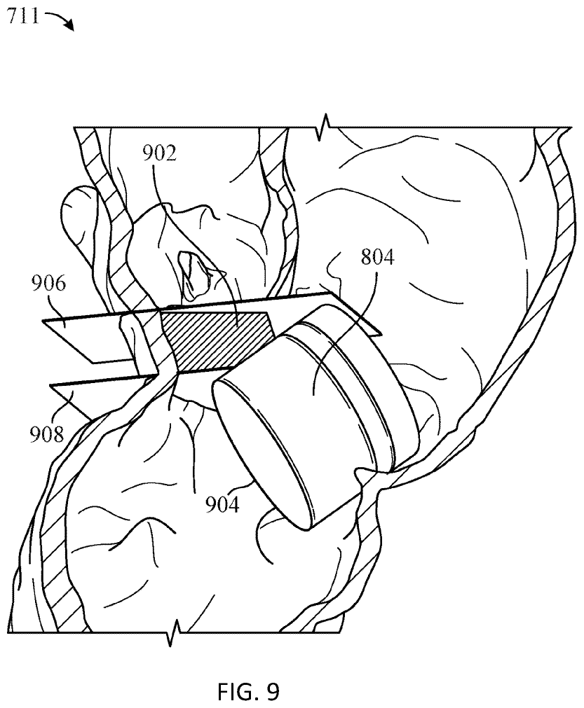

FIG. 9 is a sectional view of a structure of interest 711 from the 3D model 602 of FIG. 6. In this example, the graphic representation of the structure of interest 711 has been adapted to include a 3D virtual model of a prosthetic device 804 (in this example, a prosthetic mitral valve). Here, a region of interest 902 has been determined and is visually incorporated into the structure of interest 711. In this example, the region of interest 902 lies between a first plane 906 through the aortic valve and a second plane 908 tangential to an inferior edge 904 of the prosthetic device 804. Second plane 908 may be parallel to first plane 906.

Computing device 400 may be used to determine the region of interest 902 using the displayed structure of interest 711 with prosthetic device 804. In some examples, region of interest 902 may be visually determined by a user and/or determined by an algorithm. For example, the region of interest 902 may be indicated manually by the user on the depiction of the structure of interest 711 (e.g., prior to incorporation of the prosthetic device as shown in FIG. 7) or on the adapted depiction of the structure of interest (e.g., after incorporation of the prosthetic device as shown in FIGS. 8 and 9). In another example, the region of interest may be determined automatically by an algorithm based on, for example, manually or algorithmically identified anatomical structures in the structure of interest 711 and/or surrounding areas. For example, first plane 906 may be determined as the best-fit plane through the annulus of the aortic valve. The annulus may be indicated manually by a user on one or more depictions of the structure of interest 711, or may be detected automatically using any suitable feature-recognition technique known in the art.

The region of interest 902 may encompass the entire structure of interest 711, or only a part of the structure where the obstruction of the fluid flow may be expected. In some examples, the region of interest 902 may include the LVOT 106a and/or the neo-LVOT 106b/106c, which together are confined by the aortic valve, the basal septum, the intervalvular fibrosa, and the deflected anterior mitral valve leaflet. In some examples, the deflected anterior mitral valve leaflet may be approximated by the user or algorithm by identifying the outer wall of the representation of the prosthetic device. In some examples, the region of interest 902 may encompass the entire left ventricle, optionally excluding the volume occupied by the 3D virtual model of a prosthetic device 804.

One example of a process for determining the region of interest 902 may include the following steps: (i) a user may utilize image processing module 308 or measurement and analysis module 320 (e.g., Mimics application from Materialise NV, 3D Slicer, etc.) on computing device 400 to detect and indicate structural elements (e.g., aortic valve) of a 2D image, a set of 2D images or a 3D/4D model, either manually or utilizing 2D and/or 3D feature-recognition algorithms with or without an initial visual identification of structural elements by a user (e.g., a user may provide an initial indication of what structural aspects, such as the aortic valve annulus, correspond to which portions of the model or image(s)); (ii) upon recognition of the structural aspects, an algorithm may insert the first plane 906 through the aortic valve of the displayed structure of interest 711; (iii) (a copy of) the first plane 906 may be translated into the left ventricle until it is tangential with an inferior edge 904 of the prosthetic device 804. In this example, the translated first plane is displayed on the structure of interest 711 as a second plane 908. In this case, the region of interest 902 may be limited to the section of an adapted blood pool volume (e.g., the blood pool volume minus the volume of the representation of the prosthetic device 804) between the aortic valve or the first plane 906 and the second plane 908. In some embodiments, steps (i) and/or (ii) may be replaced by the user manually indicating the first plane 906. For example, the user may indicate three points on the structure of interest 711 (e.g., the aortic cusps) through which the first plane may be drawn. In some embodiments, step (iii) may be replaced by the user manually indicating the second plane 908. For example, the user may indicate three points on the structure of interest 711 and/or the 3D virtual model of prosthetic device 804 (e.g., a point on the inferior edge of prosthetic device 804) through which the second plane may be drawn.

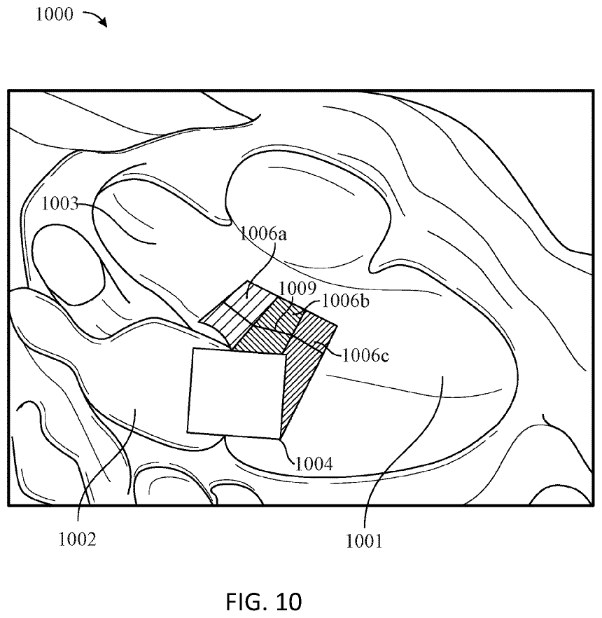

In some embodiments, the region of interest 902 may extend into the left atrium to include more than the LVOT and neo-LVOT (e.g., the neo-LVOT extension 106c of FIG. 1). FIG. 10 is a sectional view of an example 2D CT image of a patient's heart 1000. In this example, the 2D CT image includes a sectional view of the left ventricle 1001, the left atrium 1002, the aorta 1003, a virtual model of a prosthetic device 1004 (e.g., note that the virtual model is a 2D model for implementation in a 2D image), an LVOT 1006a, a neo-LVOT 1006b, an extension 1006c of the neo-LVOT, and a centerline or guide curve 1009 of the LVOT 1006a, the neo-LVOT 1006b, and the extension 1006c. In this example, the region of interest (e.g., the combination of the LVOT 1006a, the neo-LVOT 1006b, and the extension 1006c) may further include a volume that is bound by a plane that is tangential with an inferior edge 904 of the representation of the prosthetic device 804 on the posterior side. Determining the boundaries of extension 1006c may be performed on a 2D image, a set of 2D images or a 3D/4D model in much the same way as has been described above for second plane 908.

Other boundaries may also be defined. For example, a 2D or 3D guide curve 1009 through (part of) the structure of interest, including or excluding the volume occupied by the representation of the prosthetic device 1004, may be indicated by the user or may be calculated by an algorithm. For example, the guide curve may be calculated as a centerline through the left ventricle and the aorta. That is, in some embodiments, a centerline of a flow path may be calculated using the boundaries of the structure of interest, such as the lumen of the vessel through which fluid or air flows, to determine the center of the flow path. In this example, the centerline may be bounded by the intervalvular fibrosa and the basal septum, meaning that the centerline is equidistant to both structural elements. For obtaining a 2D guide curve, a 2D centerline may be calculated based on a 2D representation of the structure of interest (e.g., a 2D medical image). For obtaining a 3D guide curve, a 3D centerline may be calculated based on a 3D representation of the structure of interest (e.g., a 3D model, such as a surface model). As discussed above, performing these steps in 3D will yield more reliable results, as it allows taking the full three-dimensional shape of the anatomy into account. Next, a plane perpendicular to this guide curve 1009 may be determined as the boundary opposite the fluid flow exit (e.g., the aortic valve) of the structure of interest. In one example, this may be a plane perpendicular to the guide curve 1009 and tangential to an inferior edge of the representation of the prosthetic device 1004 on the side nearest the fluid flow exit (e.g., the anterior side). In another example, this may be a plane perpendicular to the guide curve 1009 and tangential to an inferior edge of the representation of the prosthetic device 1004 on the side farthest the fluid flow exit (e.g., the posterior side). In yet another example, this may be a plane perpendicular to the guide curve 1009 at another location (e.g., tangential to the native mitral valve annulus on the anterior or posterior side).

In some embodiments, calcifications and/or pre-existing hardware are subtracted from the blood pool volume or part of the blood pool volume to determine the region of interest. Thus, a region of interest (e.g., an LVOT 1006a, a neo-LVOT 1006b, and/or an extension 1006c) may be defined mentally by the user, or may be stored as a separate virtual entity or collection of entities. In some examples, the region of interest is stored as a virtual 3D model, such as a virtual 3D surface model (e.g., as a virtual 3D surface model of the anatomical structure of the LVOT 1006a, the neo-LVOT 1006b, and/or the extension 1006c). In other examples, the region of interest may be stored as one or more planes, curved surfaces, polygons, triangles, lines, curves, splines, cylinders or other geometrical primitives delimiting a section of the structure of interest. In some embodiments, the region of interest and/or its limits may be visualized instead of, in addition to or overlaid onto the adapted depiction. Visualization of the region of interest is not essential, but may improve the user experience, as it may make it easier for a user to form a mental image of the intricacies of the anatomy and the impact of the prosthetic device on the flow path.

All of the methods for determining a region of interest within the structure of interest described above may be processes performed by a user of computing device 400, automatically by computing device 400, or semi-automatically by computing device 400 using some input from a user.

### Defining a Guide Curve Through the Region of Interest

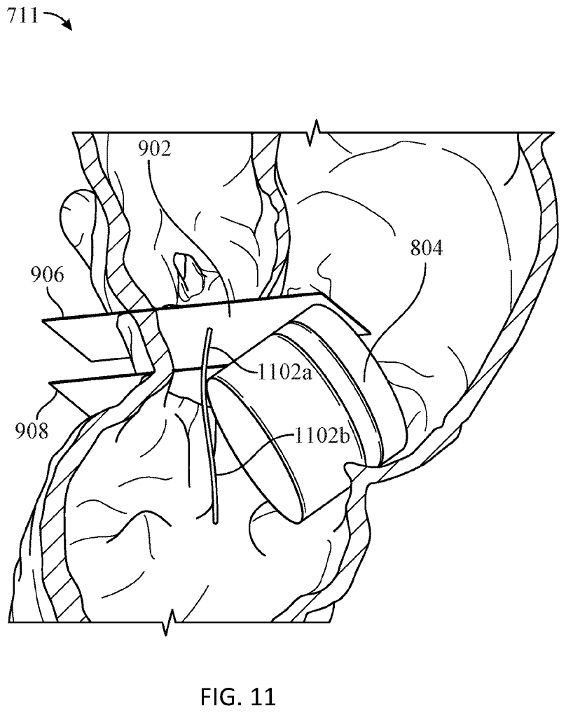

FIG. 11 is a sectional view of a structure of interest 711 from the 3D model 602 of FIG. 6. In this example, a guide curve 1102 has been determined and visually adapted to the structure of interest 711. Here, the guide curve 1102 includes a first portion 1102a being a centerline of the region of interest (e.g., the LVOT 1006a and the neo-LVOT 1006b of FIG. 10), and a second portion 1102b being a centerline of an extension of the region of interest (e.g., the extension 1006c of FIG. 10). In some examples, a location and direction of a guide curve 1102 may be determined, and a graphic representation of the guide curve 1102 incorporated into the depiction of the structure of interest 711 (e.g., prior to incorporation of the prosthetic device) or on the adapted depiction of the structure of interest (e.g., after incorporation of the prosthetic device). Guide curve 1102 may be determined based on characteristics of the region of interest (e.g., the guide curve may include a centerline of the region of interest). Centerlines may be calculated automatically using any suitable centerline algorithms known in the art. Robustness of certain of these algorithms may be improved by applying them on an extended region of interest (e.g., incorporating the neo-LVOT extension 1006c, or extending even further into the left ventricle or into the aorta).

In another example, the guide curve 1102 may be generated between two points selected by the user or an algorithm. For example, the user may arbitrarily select two points on a displayed structure of interest 711. Alternatively, an algorithm may select two points at opposite ends of the structure of interest 711 or region of interest. In this case, the algorithm may select the two points such that both points are centered in a fluid or air flow path (e.g., in this example, the left ventricle). In other examples, a first point may be: (i) an arbitrary point in the structure of interest 711, (ii) a manually indicated point in the structure of interest 711, (iii) an anatomical landmark of the structure of interest 711 (e.g., the apex of the left ventricle), or (iv) the center of the structure of interest 711 or the region of interest, either including or excluding the volume of the representation of the prosthetic device 804 (e.g., the center of gravity of the blood pool volume of the left ventricle, optionally excluding the volume of the prosthetic device 804 in its planned position, any calcification, and/or any pre-existing hardware). A second point may be a point near the fluid flow exit of the structure of interest 711, such as an arbitrary point near the aortic valve, a manually indicated point near the aortic valve, or an automatically determined point, such as the center of the aortic valve. In some examples, multiple points, such as control points or points on the guide curve, may be added between the first and second points to provide the user with more control over the direction of the guide curve. In some examples, the guide curve may comprise one or more straight-line segments, and/or one or more curved segments, such as spline curves, between the selected points. In some examples, the guide curve is defined such that its direction near the fluid flow exit is substantially parallel to the fluid flow path (e.g., perpendicular to the aortic valve or a best-fit plane through the aortic valve).

In some examples, guide curve 1102 is manually defined by a user (e.g., the user may hand-draw the guide curve on the depiction or the adapted depiction). The user may include a medical professional or a non-medical professional, such as an engineer or a technician, or any other suitable user. Alternatively, guide curve 1102 is determined algorithmically, e.g., by computing device 400, based on the depiction or on the adapted depiction. For example, guide curve 1102 may be determined as a straight line between the first and second points. Alternatively, guide curve 1102 may be determined as the centerline of structure of interest 711 and/or region of interest, including or excluding the volume of the representation of prosthetic device 804. In this case, the algorithm may determine guide curve 1102 such that the guide curve 1102 follows a centerline defined by the structure of interest 711 and/or region of interest. For example, the algorithm may determine (part of) the path of the guide curve 1102 based on the walls of the aorta, such that the guide curve 1102 follows a path that is substantially centered in the blood flow path.

In some examples, instead of defining the guide curve 1102 as a centerline through the structure of interest 711 and/or region of interest, the guide curve 1102 may be defined as a straight line segment (e.g., not necessarily centered) through the structure of interest 711 and/or region of interest (e.g., along the blood flow path of the aorta). In some examples, the guide curve 1102 may be defined as a line segment (e.g., not necessarily centered or straight) through the structure of interest 711 and/or region of interest. In other examples, the guide curve 1102 may be a straight line segment or a curve, such as a spline, or a combination thereof, that substantially follows the fluid flow path from the first point to the second point.

Guide curve 1102 may be visually displayed as an object on 2D image data or on 3D/4D models. In some examples, guide curve 1102 may be a 2D curve (e.g., fully defined in one plane and graphically represented on a single 2D image, a single MPR image, or a 3D model) or a 3D object represented on a single 2D image, a single MPR image, or a 3D/4D model. Displaying guide curve 1102 is not essential but may improve the user experience.

### Defining a Plurality of Evaluation Planes

As discussed, the guide curve 1102 may be a curve or line substantially following a fluid/air flow path, or may be a centerline of (part of) the structure of interest 711 or of the region of interest. In some embodiments, one or more planes may be defined and optionally visually displayed along guide curve 1102 to determine a cross-sectional surface area of the structure of interest or region of interest. As such, the one or more planes may intersect with the surrounding anatomical structures shown in the 2D image, set of 2D images or 3D/4D model and may define closed cross sections through which fluid/air will flow. One or more planes orthogonal to the guide curve provide a good or even an optimal basis for the evaluation of fluid flow obstruction because a laminar flow can be expected to flow substantially parallel to the guide curve. Thus, determining the cross-sectional surface area of a particular region will provide a user with information indicative of whether a possible obstruction of the fluid/air flow will result from a prosthetic device.

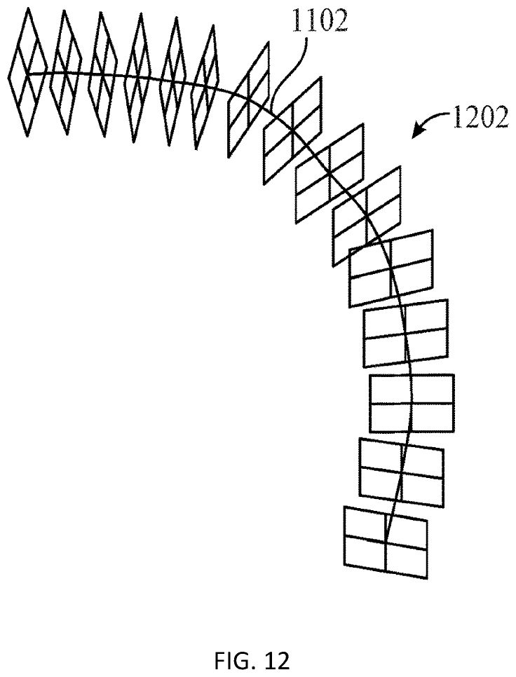

FIG. 12 is a diagram illustrating a conceptual guide curve 1102 having a plurality of planes 1202 defined orthogonally to guide curve 1102. Here, the plurality of planes 1202 are defined orthogonal to guide curve 1102 at (e.g., regular) intervals along guide curve 1102. The intervals may be determined by an algorithm based on the length of guide curve 1102, the gradient of the anatomical structure(s) along the guide curve 1102, etc. Smaller intervals may be chosen in zones where the likelihood of an obstruction is higher and vice versa. The user may also determine the intervals to strike a balance between accuracy and computational load, such as a value between 1 mm and 2 cm (e.g., 2 mm, 3 mm, 4 mm, 5 mm, 6 mm, 7 mm, 8 mm, 9 mm, 1 cm, or 1.5 cm). The plurality of planes 1202 may constitute a plurality of evaluation planes.

It should be noted that a manually drawn guide curve 1102 may be less accurate relative to an algorithmically determined guide curve 1102, and an automatically determined guide curve 1102 may not reflect all anatomical features (e.g., local protrusions or indentations) along the anatomical boundaries (e.g., walls of an aorta, a ventricle or a (neo-) LVOT, or walls of a breathing passage) of the region of interest.

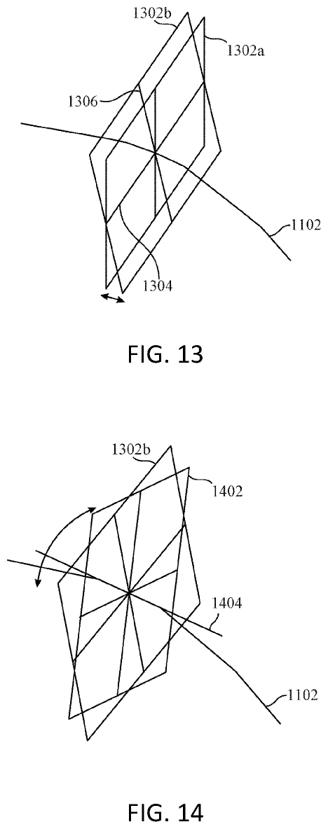

FIG. 13 is a diagram illustrating a conceptual guide curve 1102 having a first plane 1302a of the plurality of planes 1202. Note that, as illustrated in FIG. 13, the first plane 1302a refers to one of the planes 1202. Further, rotated first plane 1302b refers to a rotation of the first plane 1302a about a first axis 1304. Though not shown, additional rotations of the first plane 1302a about the first axis 1304 may be included, and each rotated position of the first plane 1302a may be added to the plurality of evaluation planes. Thus, in the context of the plurality of planes 1202 along the guide curve 1102, each of the plurality of planes 1202 are in a first position resulting in a first plurality of evaluation planes (e.g., one evaluation plane corresponding to each of the plurality of planes 1202). Yet, additional evaluation planes may be generated by incrementally rotating one or more of the plurality of planes 1202. For example, each incremental rotation of the first plane 1302a may result in an additional evaluation plane (e.g., rotated first plane 1302b).

The plurality of planes 1202 may be generated orthogonally along the guide curve 1102 and rotated about local rotation axes to provide additional evaluation planes. For example, each of the plurality of planes 1202 may be rotated about a local rotation axis in angular increments in one or more directions. In this example, the first plane 1302a may be rotated along the first axis 1304 (e.g., tangential to the first plane 1302a/locally perpendicular to the guide curve 1102) through the intersection point of the first plane 1302a and the guide curve 1102. Alternatively, or in addition, the first plane 1302a may be rotated along a second axis 1306 (e.g., tangential/locally perpendicular to the guide curve 1102 and the first axis 1304). Each plane may be rotated over one or more (e.g., fixed) angular increments, such as 1°, 2°, 5°, 10°, 12°, or 15°. After each rotation, the resulting rotated plane, e.g., plane 1302b, may be added to the plurality of evaluation planes. As such, the rotation of the first plane 1302a may result in multiple different evaluation planes.

Accordingly, the plurality of evaluation planes (e.g., a second plurality of planes) may be defined along the guide curve 1102 by rotating one or more of the plurality of planes 1202 about one or more axes tangential/perpendicular to the guide curve 1102 over one or more angular increments. In one example, one or more of the plurality of evaluation planes correspond to a first rotation of one plane (e.g., first plane 1302a) of the one or more of the plurality of planes 1202 about the first axis 1304 perpendicular to the guide curve 1102 over one or more angular increments, respectively. Thus, each of the plurality of planes 1202 and each of the plurality of evaluation planes may intersect an anatomical structure (e.g., in the region of interest) at a different location. In another example, one or more of the plurality of evaluation planes correspond to a second rotation of one plane (e.g., first plane 1302a) of the one or more of the plurality of planes 1202 about a second axis 1306 perpendicular to the guide curve 1102 and the first axis 1304. In some examples, the intersection between a plane and the anatomical structure may define a cross section of the anatomical structure, as discussed further below.

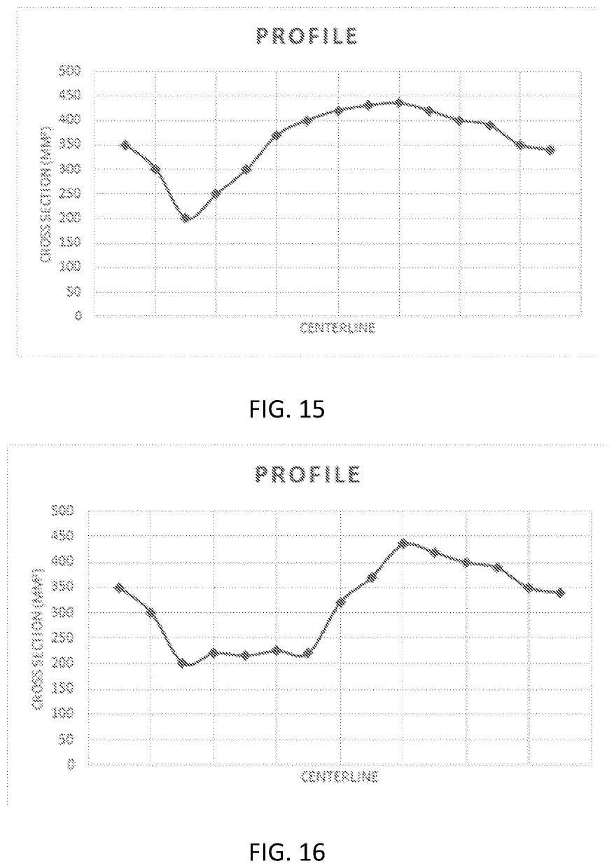

FIG. 14 is a diagram illustrating the conceptual guide curve 1102 having the rotated first plane 1302b corresponding to a rotation of one of the plurality of planes 1202. Note that, as illustrated in FIG. 14, there is the first rotated plane 1302b and a further rotated plane 1402 corresponding to a further rotation of the rotated 1302b about a different third axis 1404, that is different than the axis about which first plane 1302a was rotated to generate rotated plane 1302b. Additional rotations may be included, and each rotation of the rotated first plane 1302b may be added to the plurality of evaluation planes. Further, any one or more rotated planes generated from any one or more of planes 1202 may similarly be further rotated along a different axis and the result added to the plurality of evaluation planes. In this example, the rotated first plane 1302b may be rotated along the third axis 1404 (e.g., tangential/locally perpendicular to the guide curve 1102 and perpendicular to the first axis 1304) through the intersection point over one or more (e.g., fixed) angular increments, such as 1°, 2°, 3°, 5°, 10°, 12°, 15°, 20°, 30°, 45° or 90°, such as making full circle (e.g., 35 rotations over 10° or 17 rotations over) 20°. Accordingly, one or more of the rotations of one or more of the plurality of planes 1202 may be further rotated along a corresponding third axis 1404. Additional evaluation planes may be defined by combining rotations over zero or more angular increments over the three axes. In some examples, each rotational process may be followed by one or more additional rotational processes, where each plane is rotated in a different direction during each process. In one example, rotation of a plane about the third axis 1404 may be performed on a plane only after a rotation of that plane about the first axis 1304, the second axis 1306, and/or any additional axes. After each rotation process, the resulting planes are added to the plurality of evaluation planes.

In some examples, 3D measurement and analysis module 320 of FIG. 3 may perform the generation of the plurality of planes 1202 and guide curve 1102, as well as any plane rotation and cross-sectional measurements of each plane. By extending the plurality of evaluation planes using rotations of the planes, local inaccuracies of a manually indicated or automatically computed guide curve can be overcome, and the chance of finding the true smallest cross section through the region of interest can be improved.

### Determining a Cross-Sectional Size

In some embodiments, a surface area of a cross section of an anatomical structure (e.g., in the region of interest) where each of the plurality of planes 1202 intersects with the anatomical structure may be determined. Cross sections may be determined of the native anatomical structure, or, e.g., when the impact of a restricting or obstructing element, such as a prosthetic device, on the fluid/air flow is to be evaluated, the presence of such an element may be taken into account in the determination of cross sections. In one example, an algorithm may generate a segment image or shape (e.g., as a polygon, a closed polyline, or closed curve) of the intersection between each plane and the lumen of the anatomical structure through which fluid/air will flow, and then calculate the surface area of that cross section. The same may be done for each angular increment of rotation of the plane, e.g., for each plane of the plurality of evaluation planes.

For example, in an adapted depiction of a 2D image, a user or algorithm may calculate a distance from one end of where a plane intersects the anatomical structure or the virtual model of a prosthetic device, to another end of where the same plane intersects the other side of the anatomical structure or the virtual model of a prosthetic device. In another example, the adapted depiction of the structure of interest may include a 3D surface model of the region of interest, and the surface area of the cross section of the region of interest at an evaluation plane may be determined by first determining the intersection of the evaluation plane with the anatomical structure of the 3D surface model, and by subsequently determining the surface area of that cross section. In some examples, the determination of a surface area of a cross section of a 3D model may be performed by an algorithm using 3D geometry operations.

Optionally, one or more evaluation planes may be discarded based on their position with respect to: (i) the representation of the prosthetic device, (ii) the structure of interest, (iii) the region of interest, or (iv) the boundaries of any of these entities. For example, as blood can be expected to flow perpendicularly to the aortic valve, all evaluation planes that intersect the aortic valve may be discarded. In another example, as fluid flow obstruction due to the placement of the prosthetic device is evaluated, all evaluation planes that do not intersect the representation of the prosthetic device may be discarded.

In some embodiments, the smallest cross section is determined by finding the smallest surface area among all, or remaining, evaluation planes (e.g., among each of a plurality of planes and each of their rotations). This smallest surface area may be interpreted as a measure for the passageway still available for fluid/air flow after the planned medical procedure, or as a measure of a level of obstruction that will result from the planned medical procedure. The smallest surface area may be determined via an algorithm or manually by a user.

In some examples, the determination of the smallest cross section may allow a user to approve, reject, or modify a pre-planned treatment. The methods described herein may be repeated over a plurality of virtual treatment plans in order to compare and select an optimal treatment plan (e.g., to select the type and size of prosthetic device and/or the position of the prosthetic device best suited for a patient).

In some examples, the plurality of planes defined along the guide curve may be divided into a plurality of subsets, each of the subsets comprising those one or more planes that share an intersection point with the guide curve. In this example, a smallest cross section among the one or more planes in a subset is determined for each subset. Accordingly, multiple cross sections may be used to evaluate fluid/air flow in the structure of interest and/or region of interest. For example, all evaluation planes that share the same intersection point with the guide curve can be grouped in one subset (e.g., all evaluation planes that are generated by rotating a first plane about one or more axes and/or points). As such, the evolution of the minimum cross-sectional surface area may be followed along the guide curve.

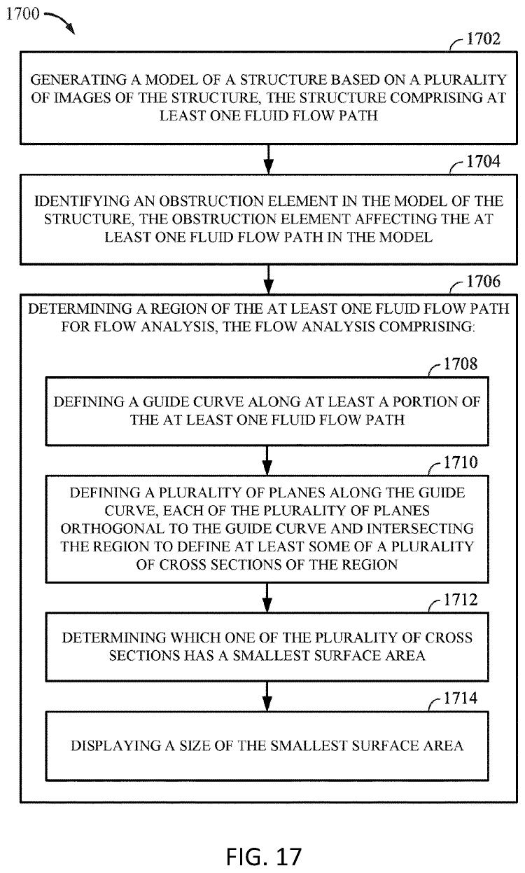

In some examples, a smallest surface area of each of two or more subsets along the guide curve may be graphically profiled to generate a smallest surface area profile. This provides the benefit of being able to distinguish between a discrete stenosis (e.g., a localized narrowing of the passageway) and a tunnel stenosis (e.g., a narrowing over a relatively longer distance). The multiple cross section surface areas may be graphically profiled and presented as a graph (e.g., where the distance along the guide curve is presented along the horizontal axis, and the size of the cross section of the anatomical structure is presented along the vertical axis). For example, both FIGS. 15 and 16 are example graphs illustrating a cross-sectional area along a centerline or guide curve of a fluid or air passageway. In this example, FIG. 15 may indicate a discrete stenosis, while FIG. 16 may indicate a tunnel stenosis.

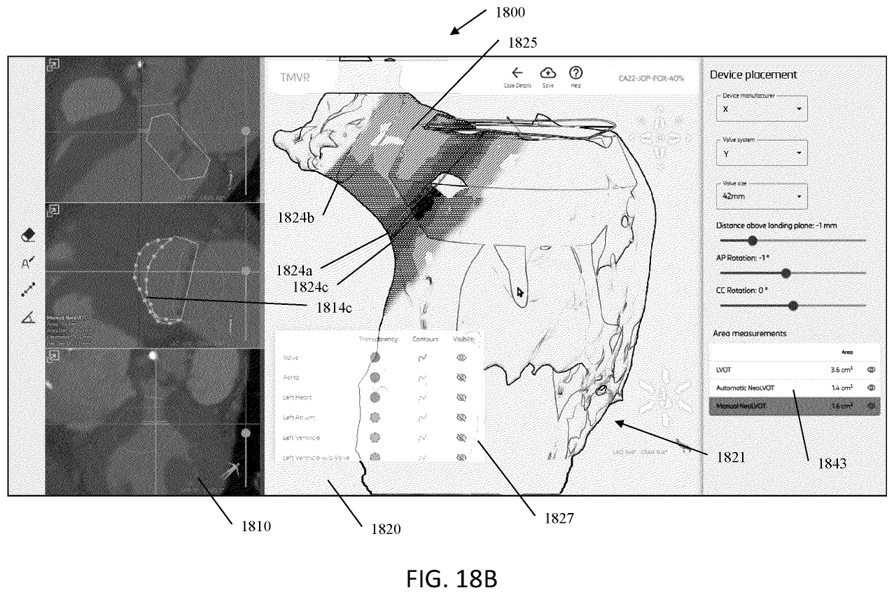

Alternatively or additionally, cross-sectional surface areas of an anatomical structure may be graphically presented in the adapted depiction. For example, a false-color overlay on a structure of interest and/or a region of interest indicating cross-sectional sizes may be displayed, where the colors of the surfaces of the 3D model are mapped to different cross-sectional sizes. This is elaborated further below in the context of FIG. 18B.

As noted above, the techniques described herein may also be used to identify a type of stenosis in an anatomical structure of a patient. In one example, a threshold value may be determined for the number of evaluation plane subsets and/or the length of the section of the guide curve along which a narrowing may be identified. A narrowing below this threshold (e.g., a small number of cross-sectional sizes in one region that are relatively smaller than other cross-sectional sizes outside of the one region) may be identified as a discrete stenosis. A narrowing above this threshold (e.g., a large number of cross-sectional sizes in one region that are relatively smaller than other cross-sectional sizes outside of the one region) may be identified as a tunnel stenosis. That is, a relatively localized or short distance of small cross-sectional sizes will likely correspond to a discrete stenosis, whereas a relatively broader distance of small cross-sectional sizes will likely correspond to a tunnel stenosis.

In some examples, cross-sectional values for both the pre-procedural (e.g., using a visual depiction of a structure of interest prior to procedure) and post-procedural situation (e.g., using a visual depiction of the structure of interest adapted with the post-procedure prosthetic device) may be calculated and presented to the user. Here, the smallest cross section may be determined in a pre-procedural situation (e.g., the smallest cross section is determined in an original depiction of an anatomical structure, not an adapted depiction). This may result in the smallest cross section of the LVOT. Then, the smallest cross section may be determined in a post-procedural situation (e.g., the smallest cross section is determined in an adapted depiction). Systems according to certain embodiments may then display the ratio of the post-procedural smallest cross section and the pre-procedural smallest cross section as a measure for the remaining fluid/air passageway, or as a measure of obstruction of the fluid/air passageway. For example:

In some examples, the system may display a ratio of a determined smallest cross section of the region of interest and a cross section of the pre-procedural structure in the same evaluation plane.

### User Interface

In the following section, the word “user” relates to a human user of computer environment 300 and/or computing device 400. The user may be a medical professional or a non-medical professional, such as a technician or engineer.

As described above, computer environment 300, image processing module 308, 3D measurement and analysis module 320 and/or a single module combining the functionalities of both image processing module 308 and 3D measurement and analysis module 320 may comprise a user interface. FIG. 18a shows an example of such a user interface 1800 that may improve the user experience. In the following paragraphs, references to computer environment 300 should be understood to refer to one or more of computer environment 300, image processing module 308, 3D measurement and analysis module 320 and/or a single module combining the functionalities of both image processing module 308 and 3D measurement and analysis module 320.

User interface 1800 may comprise one or more areas presenting different functionality. For example, user interface 1800 may comprise one or more image-display areas 1810 for displaying one or more 2D (e.g., medical) images, multi-planar reconstruction (MPR) images and the like. In the example shown, user interface 1800 comprises three image-display areas 1810, each displaying one MPR image of a structure of interest, here a left ventricle of a patient. The MPR images may be generated by means of any suitable techniques known in the art from previously acquired images, such as CT scans, and may each show one slice through the structure of interest in a viewing direction perpendicular to the viewing directions of the other MPR images. Image-display areas 1810 may offer functionality such as zooming, panning and scrolling through the slices in the viewing direction. Cross hairs 1811 in one image-display area may indicate the slice positions shown in the other image-display areas. One or more geometric entities, such as indicated landmarks (e.g., an aortic valve annulus or a mitral valve annulus), annotations, cross sections of virtual models inserted into the model of the structure of interest, measurements and the like may be overlaid onto the displayed 2D image(s). Here, a cross section 1814b through the LVOT and a cross section 1813 of a virtual model of a prosthetic valve are displayed.

User interface 1800 may further comprise one or more 3D-display areas 1820 for displaying one or more 3D renderings and/or virtual 3D models. In the example shown in FIG. 18a, user interface 1800 comprises one 3D-display area 1820, displaying an adapted model 1821 of the structure of interest, here comprising a virtual surface model 1822 of the blood pool volume of a patient's left ventricle and a virtual 3D model 1823 representing a prosthetic mitral valve. One or more geometric entities, such as indicated landmarks, annotations, cross sections of virtual models inserted into the model of the structure of interest, measurements and the like may be overlaid onto the displayed 3D renderings and/or 3D models or the model of the structure of interest may be further adapted to comprise such geometric entities. Here, the adapted model 1821 further comprises a cross section 1824b through the LVOT and a cross section 1824a through the neo-LVOT.

3D-display areas 1820 may offer all common 3D viewing capabilities, such as zooming, panning, rotating, clipping, choosing transparency levels of displayed models or choosing between parallel or perspective viewing. These viewing capabilities may be controlled through any suitable I/O device(s), such as keyboard keys, mouse buttons, mouse scroll wheels, touch screens or through controls displayed in user interface 1800, such as controls 1828. A 3D-display area 1820 may further offer the user the possibility to select default viewpoints, such as through controls 1829. A default viewpoint may relate to any combination of a predefined viewing direction, zoom factor and position of the displayed 3D renderings and/or virtual 3D models within 3D-display area 1820. In the example shown, a default viewpoint may be selected by clicking one of the six arrows of controls 1829. In some examples, the default viewpoints may be one or more of a top viewpoint, a bottom viewpoint, a front viewpoint, a back viewpoint, a left viewpoint and a right viewpoint. These viewpoints may relate to directions parallel to the axes of an internal orthogonal coordinate system of computer environment 300. In some examples related to patient anatomy, the default viewpoints may relate to the patient's anatomical coordinate system (e.g., computer environment 300 may align its internal coordinate system with the patient's anatomical coordinate system, or with the coordinate system of the medical images). For example, the top and bottom viewpoints may relate to craniocaudal and caudocranial (or axial) viewing directions, the front and back viewpoints may relate to anteroposterior and posteroanterior (or coronal) viewing directions, and the left and right viewpoints may relate to left and right transverse (or sagittal) viewing directions, respectively. It greatly improves efficiency and user friendliness if the default viewpoints relate to the structure of interest. For example, in examples where the structure of interest is a heart or a left ventricle, computer environment 300 may calculate and offer default viewpoints that differ from the patient's anatomical coordinate system and offer the user a better view of the heart's or the left ventricle's anatomical parts. For example, the front and back viewpoints may correspond to substantially anteroposterior and posteroanterior viewing directions, respectively, within a plane/planes parallel to the mitral valve and/or the aortic valve. For example, the top and bottom viewpoints may correspond to substantially craniocaudal and caudocranial viewing directions, respectively, perpendicular to the mitral valve. For example, the left and right viewpoints may correspond to substantially left and right transverse viewing directions, respectively, perpendicular to the top, bottom, front and back viewpoints' viewing directions. In this context, “parallel to a valve” or “perpendicular to a valve” may respectively mean parallel or perpendicular to a best-fit plane through (part of) said valve, or to a best-fit plane through (part of) said valve's annulus. For example, after data acquisition, the user may indicate or computer environment 300 may automatically recognize certain anatomical landmarks, such as the left ventricle, the mitral valve annulus and/or the aortic valve annulus, and/or a region of interest, such as an LVOT, a neo-LVOT and/or an extension of the neo-LVOT (e.g., as part of any of the processes disclosed herein). Based on these anatomical landmarks, computer environment 300 may reorient its default viewpoints to a coordinate system that relates to the structure of interest, as illustrated above for the left ventricle.

3D-display areas 1820 may offer the user the possibility to selectively display or hide certain 3D renderings, virtual 3D models and/or any of the geometric entities described above. For example, as is shown in FIG. 18B, pop-up menu 1827 may allow the user to control for each of these elements whether it is shown, the transparency level of its depiction, and/or whether contours (e.g., edges of surfaces, sharp creases . . . ) are shown.

Continuing with FIG. 18A, user interface 1800 may further comprise an area 1830 with tools, such as annotation tools and measurement tools. These tools may allow the user to measure distances or angles, to add annotations, or to indicate anatomical landmarks in any of the image-display areas 1810 or 3D-display areas 1820.

User interface 1800 may further comprise a treatment-planning area 1840 with functionality for creating a (virtual) treatment plan. The controls in treatment-planning area 1840 may be for general-purpose functionality, such as inserting a virtual representation of a device into the model of the structure of interest, sectioning off parts of the model of the structure of interest, translating and/or rotating virtual models and the like. In some examples, the controls in treatment-planning area 1840 are adapted to the planning of a particular type of treatment. In the example shown, the controls allow the user to plan a TMVR. Controls 1841a, 1841b, 1841c allow the user to insert a virtual representation of a prosthetic mitral valve into the model of the structure of interest. As described above, the virtual representation of a prosthetic device may include a schematic representation, such as a cylinder, a truncated cone, a spindle, a double truncated cone or a combination of one or more such or other primitive shapes, and/or a more lifelike 3D model of the actual device. Additionally or alternatively, it may be or include a parameterized virtual object, a model loaded from a file, or a model loaded from a library of device templates. It may be or include a model based on a CAD design of the prosthetic device, on a (e.g., optical) scan of a physical device or on a segmentation of one or more (e.g., medical) images comprising a depiction of a prosthetic device. In the example shown, the user may select a prosthetic device by selecting a brand (control 1841a), type (control 1841b) and size (control 1841c). Treatment-planning area 1840 may further comprise controls to control the position of the inserted device with respect to the structure of interest, e.g., the planned position for the prosthetic device in the anatomical structure of the patient. Changes to the position of the inserted device with respect to the structure of interest made by means of these controls may be automatically reflected in the image-display areas 1810 and/or 3D-display areas 1820. For example, treatment-planning area 1840 may comprise controls for one or more of the six degrees of freedom (3 for translation, 3 for rotation) relative to the internal coordinate system. To improve user friendliness, the degrees of freedom may be relative to a coordinate system related to the structure of interest, or to any previously identified anatomical landmarks of said structure of interest. In the example shown, the translation is expressed relative to a best-fit plane through (part of) the mitral valve annulus, and rotation is expressed relative to the viewing directions of the default viewpoints as described above. To further improve user friendliness, the degrees of freedom that the user can control may be restricted to those that are relevant to the type of treatment being planned. In the example shown, only one translational degree of freedom is available to the user: translation in a direction perpendicular to a best-fit plane through (part of) the mitral valve annulus (control 1842a). This direction corresponds to the substantially craniocaudal direction as described above in the context of default viewpoints. Also in the example, only two rotational degrees of freedom are available to the user: rotation about a substantially anteroposterior axis parallel to the best-fit plane through (part of) the mitral valve annulus (control 1842b), and rotation about an axis parallel to said substantially craniocaudal direction (control 1842c). The other degrees of freedom may be automatically fixed by computer environment 300. For example, the translational degrees of freedom perpendicular to said substantially craniocaudal direction may be automatically fixed such that in a plane perpendicular to said substantially craniocaudal direction, the virtual representation of the device is centered with respect to the mitral valve annulus. Similarly, the remaining rotational degree of freedom may be automatically fixed such that a top or a bottom plane of the virtual representation of the device remains parallel to a substantially anteroposterior axis parallel to a best-fit plane through (part of) the mitral valve annulus. Here, the geometric center of the mitral annulus may be used as center of rotation for the rotational degrees of freedom. Other coordinate systems or selections of fixed and controllable degrees of freedom may be made for TMVR without diverging from the teachings of this disclosure. A skilled person will readily understand that applications other than TMVR may require different coordinate systems or selections of fixed and controllable degrees of freedom. A skilled person will readily understand that other embodiments may utilize other types of user controls that the ones shown in FIG. 18A-B (e.g., pop-up menus, mouse controls, controls within image-display areas 1810 or 3D-display areas 1820, etc.) without diverging from the present teachings.

User interface 1800 and/or treatment-planning area 1840 may further comprise one or more interface elements 1843 for reporting information related to the treatment plan to the user. This may be information to allow the user to evaluate the treatment plan. In some examples, the information may relate to a degree of obstruction in a fluid or air passageway of the structure of interest. For example, interface elements 1843 may report the smallest cross-sectional surface area through the fluid/air passageway after introduction of a planned prosthetic device. Computer environment 300 may calculate said smallest cross-sectional surface area using any of the methods disclosed herein. For example, with reference to the previous sections, optionally, a region of interest may be determined manually or automatically by computer environment 300 as described above. Optionally, a guide curve through the region of interest may be determined as described above. A plurality of evaluation planes may be determined as described above, optionally based on said guide curve. Next, computer environment 300 may determine a smallest cross-sectional size among the plurality of evaluation planes as described above. More particularly, where the treatment plan relates to TMVR, interface elements 1843 may report the smallest cross-sectional surface area through the LVOT, neo-LVOT and/or extension of the neo-LVOT as a measure of blood-flow obstruction. This will allow a user to assess the impact of the planned procedure on the fluid/air flow through the passageway. For example, if the reported smallest cross-sectional surface area through the LVOT/neo-LVOT/extension of the neo-LVOT drops below a predetermined threshold, the user may decide to reject the treatment plan, or to adapt the treatment plan, e.g., by selecting a different type and/or size of prosthetic device, and/or by altering the planned position of the prosthetic device. In some examples, interface elements 1843 may report further information. In the example shown, interface elements 1843 report three cross-sectional areas: a smallest cross-sectional area through the LVOT in a native, pre-procedural state, an automatically determined smallest cross-sectional area through the LVOT/neo-LVOT/extension of the neo-LVOT, and a cross-sectional area through the LVOT/neo-LVOT/extension of the neo-LVOT along a section plane indicated by the user. In the example of FIG. 18A, the user has not yet manually indicated a section plane, and the automatically determined smallest cross-sectional area through the neo-LVOT/extension of the neo-LVOT is reported instead. In FIG. 18B, the user has manually indicated a section plane. Interface elements 1843 may offer additional functionality. For example, where the reported information relates to a measurement, the location where the measurement was taken may be depicted in one or more image-display areas 1810 and/or 3D-display areas 1820. Selecting an information in interface elements 1843 may cause the depiction(s) of the corresponding measurement location to be highlighted in display area(s) 1810, 1820. In the example shown, the reported cross-sectional surface areas correspond to cross sections 1824a, 1824b, 1824c through the LVOT/neo-LVOT/extension of the neo-LVOT along section planes. These cross sections are displayed in 3D-display area 1820. The smallest cross-sectional area through the LVOT in a native, pre-procedural state corresponds to cross section 1824b, the automatically determined smallest cross-sectional area through the LVOT/neo-LVOT/extension of the neo-LVOT corresponds to cross section 1824a, and the cross-sectional area through the LVOT/neo-LVOT/extension of the neo-LVOT along a section plane indicated by the user corresponds to cross section 1824c. In FIG. 18A, cross section 1824b is also depicted as cross section 1814b in one of the image-display areas 1810. In FIG. 18B, cross section 1824c is also depicted as cross section 1814c in one of the image-display areas 1810. Highlighting a cross section in a 3D-display area 1820 or an image-display area 1810 may comprise, e.g., one or more of giving its contour a bright or contrasting color, increasing its contour's thickness or filling in the area within its contour with a bright or contrasting color. Additionally or alternatively, highlighting a cross section in an image-display area 1810 may further comprise, e.g., changing the reconstruction directions of one or more MPR images and/or displaying a slice coincident with the section plane of the cross section. This greatly improves user friendliness, as it helps the user get a visual impression of the passageway at the location of the cross section. For example, in FIG. 18A, the images shown in image-display areas 1810 have been reconstructed such that the middle image-display area shows an image coinciding with the section plane of cross section 1814b and the top and bottom image-display areas show images in view directions perpendicular to said section plane. Similarly, in FIG. 18B, the images shown in image-display areas 1810 have been reconstructed such that the middle image-display area shows an image coinciding with the section plane of cross section 1814c and the top and bottom image-display areas show images in view directions perpendicular to said section plane.

As described above, in some examples, the treatment plan may be created automatically, e.g., by computer environment 300 or computing device 400, based on automatically identified anatomical structures and/or anatomical structures previously identified by a user, such as a medical professional, or a non-medical professional, such as a technician or engineer, and/or based on a planning algorithm dictating prosthetic device selection and/or position. In some examples, the device size selection and/or the selection of the position (e.g., location and orientation) of the device is made with respect to the patient's anatomy: for example, with respect to features of the patient's anatomy, to anatomical landmarks, to geometrical primitives, such as coordinate systems, planes, lines, vectors or points, derived from such features or landmarks, or to measurements taken of or among such features, landmarks or geometrical primitives. To facilitate device selection, computer environment 300 may comprise a library of devices, e.g., in image data storage 306, in image processing module 308 or 3D measurement and analysis module 320. Each entry in the library may represent a device of a particular brand, type and/or size. The library may comprise for each entry a virtual representation of a device, such as any of the virtual representations mentioned above. One or more characteristic specifications of the device, such as a height, a radius, a diameter and/or a local coordinate system, may also be stored as part of the library entry. These characteristic specifications may be used by algorithms to select a device (e.g., brand, type and/or size) and/or its position for an automatically created treatment plan. In some examples, a partial or full treatment plan may first be created automatically. For example, a device type and/or size may first be determined automatically based on features of the patient's anatomy, anatomical landmarks, geometrical primitives, such as coordinate systems, planes, lines, vectors or points derived from such features or landmarks, or on measurements taken of or among such features, landmarks or geometrical primitives. For example, based on a model of the structure of interest, e.g., a left ventricle, including a mitral valve, and/or an anatomical landmark, e.g., a mitral valve annulus, which has been manually or automatically identified within the model of the structure of interest, a prosthetic device, e.g., a prosthetic mitral valve, may be selected that best fits the dimensions of the structure of interest and/or the anatomical landmark. For example, an average diameter or radius of a mitral valve annulus may be determined, and subsequently a prosthetic mitral valve (e.g., brand, type and/or size) with a diameter or radius which is closest to the average diameter or radius of the mitral valve annulus may be selected. In some embodiments, the selection is limited to those prosthetic mitral valves with diameters or radii greater than the average diameter or radius of the mitral valve annulus, in order to avoid the risk of device loosening. Other device selection algorithms may be used, such as the methods described in publication WO2015/179543 A1, which is incorporated herein by reference in its entirety. In this context, the average diameter or radius of a mitral valve annulus may be determined, after the mitral valve annulus has been manually or automatically identified to create a virtual mitral valve annulus representation, by fitting a circle or ellipse through said virtual mitral valve annulus representation. Because the human mitral valve annulus may have a saddle shape, comprising a larger planar section on the lateral side and a smaller, non-planar section on the medial side, it may be beneficial to fit the circle or ellipse through only part of the virtual mitral annulus representation, e.g., only through the lateral part. Additionally or alternatively, a position (e.g., a location and/or orientation) for a device may be automatically determined. In some examples, a position is automatically determined after every automatic and/or manual device selection. For example, after fitting the circle or ellipse through (part of) the virtual mitral valve annulus, a geometric center point and a plane of said circle or ellipse may be determined. Automatically determining a position for the prosthetic mitral valve may then comprise translating and/or rotation the virtual representation of the prosthetic mitral valve until the origin of its local coordinate system coincides with the geometric center point of the circle or ellipse, and one axis of its local coordinate system is perpendicular to the plane of the circle or ellipse. Other planning algorithms may be conceived without diverging from the present teachings. After a partial or full treatment plan has been automatically created, a user may decide, e.g., based on the information reported in interface elements 1843, to alter the treatment plan (e.g., select a different device and/or change the device's position, e.g., by means of controls 1841a, 1841b, 1841c, 1842a, 1842b, 1842c). After a partial or full treatment plan has been manually or automatically created or adapted, the depictions in image-display areas 1810 and/or 3D-display areas 1820 may be updated to bring them into conformity with said partial or full treatment plan. For example, the virtual representation of the selected device may be inserted into the model of the structure of interest to create an adapted model of the structure of interest and said adapted model may be displayed in 3D-display area 1820. The position of the virtual representation of the selected device relative to the model of the structure of interest may be made to correspond to the position of the prosthetic device relative to the structure of interest in the treatment plan. It greatly benefits the user experience if these updates to the depictions in image-display areas 1810 and/or 3D-display areas 1820 are performed automatically and in real time.

User interface 1800 may comprise additional areas with user interface controls, such as title bars, menus, pull-down menus, text fields and the like, e.g., for creating a new treatment plan, for data acquisition, for loading and storing files, for displaying a patient identifier, for navigating between steps in a process flow, etc.

As mentioned above, cross-sectional surface areas of an anatomical structure may be graphically presented in an adapted depiction, such as a depiction of adapted model 1821 of the structure of interest in 3D-display area 1820. For example, a false-color overlay on a structure of interest and/or a region of interest indicating cross-sectional sizes may be displayed, where the colors of the surfaces of the 3D model are mapped to different cross-sectional sizes. With reference to the previous sections, optionally, a region of interest may be determined manually or automatically by computer environment 300 as described above. Optionally, a guide curve through the region of interest may be determined as described above. A plurality of evaluation planes may be determined as described above, optionally based on said guide curve. Next, computer environment 300 may determine a cross section through the fluid/air passageway in the structure of interest along each of the evaluation planes and a surface area of said cross section as its cross-sectional size as described above. Next, each point, pixel, voxel or surface element of the adapted depiction, as the case may be, may be colored in accordance with the cross-sectional size of its nearest cross section or with an interpolated value based on the cross-sectional sizes of its nearest cross sections. This is illustrated in FIG. 18B. A false-color overlay is depicted on a region of interest 1825, here an LVOT/neo-LVOT/extension of the neo-LVOT, in an adapted model 1821 of a structure of interest, here a left ventricle of a patient. All surface elements within the region of interest near a cross section with a cross-sectional size below a first predetermined threshold are colored red. All surface elements within the region of interest near a cross section with a cross-sectional size above a second, higher predetermined threshold are colored green. All surface elements within the region of interest near a cross section with a cross-sectional size between the first and second predetermined thresholds have a color ranging from red to green. It is understood that other color scales may be chosen. Moreover, the color mapping may be based on any number of predetermined thresholds, such as 0 or more. For example, red may be mapped to the smallest cross-sectional size found and green to the largest cross-sectional size found. Displaying a false-color overlay on a structure of interest and/or a region of interest indicating cross-sectional sizes greatly improves the user friendliness, in that it offers a user highly intuitive visual feedback regarding the suitability of the treatment plan.

In some examples, the automatically determined smallest cross section 1824a through region of interest 1825 and/or the false-color overlay on region of interest 1825 may be updated upon request by the user, e.g., after creation of the treatment plan or after one or more changes have been made to the treatment plan. In other examples, e.g., to improve efficiency and user friendliness, the automatically determined smallest cross section 1824a through region of interest 1825 and/or the false-color overlay on region of interest 1825 may be updated automatically upon creation of and after each change made to the treatment plan. To this end, computer environment 300 may repeat the processes described above for determining the smallest cross section and the false-color overlay. In preferred embodiments, both processes are combined to avoid unnecessarily repeating computational steps. For example, with reference to the previous sections, optionally, a region of interest may be determined manually or automatically by computer environment 300 as described above. Optionally, a guide curve through the region of interest may be determined as described above. A plurality of evaluation planes may be determined as described above, optionally based on said guide curve. Next, computer environment 300 may determine a plurality of cross sections through the fluid/air passageway in the structure of interest, each cross section along one of the evaluation planes and may calculate a surface area of each cross section as its cross-sectional size as described above. The cross section with the smallest cross-sectional size among the plurality of cross sections is identified as smallest cross section 1824a. Computer environment 300 may also form subsets of cross sections sharing an intersection point with the guide curve, and subsequently identify within each subset the cross section with the smallest cross-sectional size as the smallest cross section for the corresponding point along the guide curve. Next, each point, pixel, voxel or surface element of the adapted depiction may be colored in accordance with the cross-sectional size of its nearest cross section or with an interpolated value based on the cross-sectional sizes of its nearest cross sections. If cross sections have been evaluated in subsets along the guide curve, only the smallest cross section of each of the subsets may be taken into account. Efficiency and user friendliness are further greatly improved if the automatically determined smallest cross section 1824a through region of interest 1825 and/or the false-color overlay on region of interest 1825 are updated automatically upon creation of and after each change made to the treatment plan in real time. Various optimizations of the processes described herein may be utilized to reduce the computation time of these updates and approach or achieve real-time updating. For example, the region of interest may be determined and/or anatomical landmarks may be indicated only once, e.g., upon creation of the model of the structure of interest. Additionally and/or alternatively, in the step of determining a guide curve through the region of interest, a computationally intensive step of determining a centerline through the region of interest may be avoided by determining a much simpler guide curve, e.g., a guide curve between two points selected by the user or an algorithm or a guide curve of a fixed length starting and/or ending in a point selected by the user or an algorithm. For example, the user may arbitrarily select two points on a displayed structure of interest 1821. Alternatively, an algorithm may select two points at opposite ends of the structure of interest 1821 or region of interest 1825. In this case, the algorithm may select the two points such that both points are centered in a fluid or air flow path (e.g., in this example, the left ventricle). In other examples, a first point may be: (i) an arbitrary point in the structure of interest 1821, (ii) a manually indicated point in the structure of interest 1821, (iii) an anatomical landmark of the structure of interest 1821 (e.g., the apex of the left ventricle), or (iv) the center of the structure of interest 1821 or the region of interest 1825, either including or excluding the volume of the representation of the prosthetic device 1823 (e.g., the center of gravity of the blood pool volume of the left ventricle, optionally excluding the volume of the prosthetic device 1823 in its planned position, any calcification, and/or any pre-existing hardware). A second point may be a point near the fluid flow exit of the structure of interest 1821, such as an arbitrary point near the aortic valve, a manually indicated point near the aortic valve, or an automatically determined point, such as the center of the aortic valve. In some examples, multiple points, such as control points or points on the guide curve, may be added between the first and second points to provide the user with more control over the direction of the guide curve. In some examples, the guide curve may comprise one or more straight-line segments, and/or one or more curved segments, such as spline curves, between the selected points. In some examples, the guide curve is defined such that its direction near the fluid flow exit is substantially parallel to the fluid flow path (e.g., perpendicular to the aortic valve or a best-fit plane through the aortic valve). In some examples, the guide curve is defined as a straight line segment of a fixed length ending near the fluid flow exit and parallel to the flow direction near the fluid flow exit, e.g., a line segment of half the mediolateral width of the left ventricle, perpendicular to the aortic valve, starting within the left ventricle and ending in the geometric center point of the aortic valve. Additionally or alternatively, the plurality of evaluation planes may be limited to planes perpendicular to the guide curve, omitting the steps of rotating these planes along a first, second and third axis. Alternatively, the step of determining a guide curve may be omitted altogether. Instead, the plurality of evaluation planes may be defined as a plurality of parallel planes at regular intervals and perpendicular, e.g., to the general flow direction within the fluid flow path, or the flow direction near the fluid flow exit, e.g., parallel to the aortic valve. Intervals between 1 mm and 2 cm (e.g., 2 mm, 3 mm, 4 mm, 5 mm, 6 mm, 7 mm, 8 mm, 9 mm, 1 cm, or 1.5 cm) have shown to strike a balance between accuracy and computational load. Additionally or alternatively, the plurality of evaluation planes may be defined only once, e.g., upon creation of the model of the structure of interest—and thus before the creation of the adapted model of the structure of interest—or only upon creation of the adapted model, i.e., of the first treatment plan, whether manual or automatic. For clarity, no new guide curve and/or plurality of evaluation planes are, in that case, defined upon further adaptation of the treatment plan. Defining a guide curve and/or a plurality of evaluation planes only once, irrespective of the treatment plan, has the drawback that certain geometric intricacies of the adapted model may be overlooked. However, this risk has been shown to be acceptable. Moreover, additional computational efficiency may then be achieved in the calculation of cross-sectional sizes. For example, the cross-sectional size related to an evaluation plane may be calculated as the surface area of a cross section along said evaluation plane through the original model of the structure of interest 1822 minus the virtual representation of the device 1823 (if the evaluation plane intersects the device). The cross section along each evaluation plane through the original model of the structure of interest 1822 may occur, e.g., upon definition of the plurality of evaluation planes. Then, updating the cross-sectional sizes remains limited to calculating the cross sections through the virtual representation of the device 1823—which may be sped up even further by utilizing schematic representations instead of lifelike virtual 3D models for prosthetic devices—, subtracting those cross sections from the cross sections through the original model of the structure of interest 1822, and calculating the surface areas of the resulting contour. This may be limited to those evaluation planes intersecting the prosthetic device. Moreover, as all cross sections may be closed 2D contours, such as closed polylines, subtracting cross sections is a computationally less demanding task than subtracting a virtual 3D model of the prosthetic device from the model of the structure of interest and determining an all new set of cross sections along the plurality of evaluation planes. A particularly good compromise between computational speed and accuracy may be achieved by:
defining the plurality of evaluation planes prior to the creation of the treatment plan, based on a first plurality of planes orthogonal to a guide curve at regular intervals and one or more of a second, third and fourth plurality of planes obtained by rotation about a first, second and third axis, respectively, as described above;
grouping the plurality of evaluation planes in subsets according to their intersection points with the guide curve, as described above;
calculating a smallest cross-sectional size through the original model of the structure of interest 1822 among each of the subsets and reducing the plurality of evaluation planes to a single plane per subset corresponding to said smallest cross-sectional size (the smallest among this plurality of smallest cross-sectional sizes may be reported to the user as smallest cross-sectional size of the region of interest, e.g., the LVOT); and
after creation or changing of the treatment plan, calculating the cross section through the virtual representation of the device 1823 along each of the evaluation planes, subtracting said cross section through the virtual device from the cross section through the original model of the structure of interest, and calculating the resulting contour's surface area. This step may be performed for all evaluation planes (one per subset), or only for those planes intersecting the prosthetic device.

This approach takes the intricacies the fluid flow path into account, but not the potential additional irregularity introduced by the prosthetic device.

### Report, Intra-Operative Guidance

Computer environment 300, image processing module 308, 3D measurement and analysis module 320 and/or a single module combining the functionalities of both image processing module 308 and 3D measurement and analysis module 320 may be configured to generate a report regarding the (adapted) treatment plan. The report may comprise the selected prosthetic device (e.g., brand, type, size) and its position (e.g., location, orientation) relative to the structure of interest. The report may further comprise a graphical representation of the structure of interest, such as a 2D image, a set of 2D images, a virtual 3D model, such as a surface model, a virtual 4D model, or any combination thereof. The report may be a digital file or a tangible file. The report may be stored in a file, database or any other suitable medium. The report may be used for guidance during execution of the treatment. For example, a printed copy of the report may be available to the physician. Additionally or alternatively, the report may be displayed on a display in the operating room. Additionally or alternatively, the report may be displayed by an augmented-reality guidance system. For example, the graphical representation of the structure of interest may be registered onto a medical image or set of medical images captured intra-operatively, such as a transesophageal echocardiography (TEE) image, using any suitable image registration techniques known in the art. A model of the planned prosthetic device in its planned position relative to the structure of interest and/or a graphical representation of the structure of interest may then be shown in overlay onto the intra-operatively captured image(s) for visual guidance. The model of the planned prosthetic device and/or the graphical representation of the structure of interest may be shown as 2D models (e.g., as a cross section through a 3D model), 3D models (e.g., opaque, transparent and/or clipped), 4D models (e.g., opaque, transparent and/or clipped), or any combination thereof.

### Evaluating Flow Obstruction

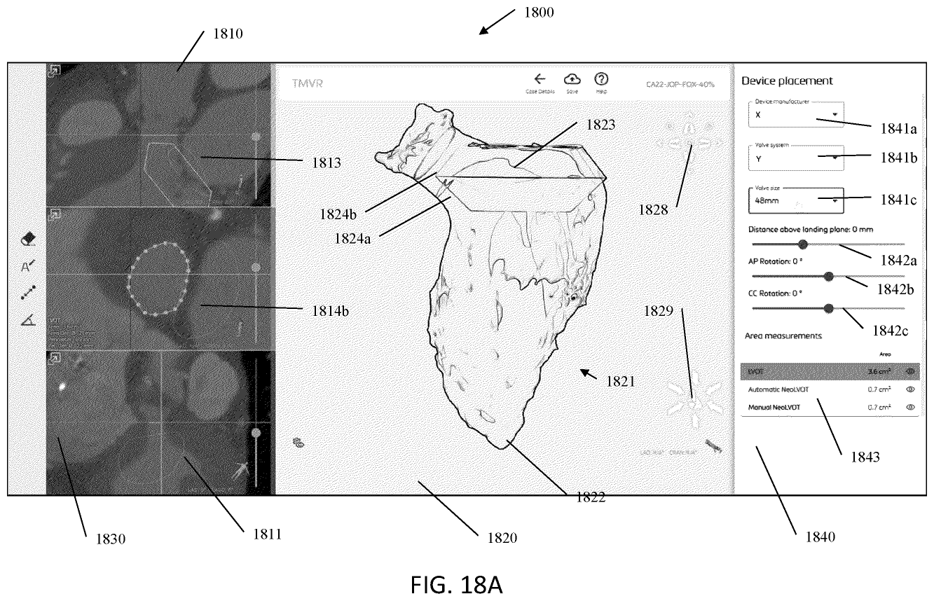

FIG. 17 is a flow chart illustrating a process 1700 for determining a minimum, e.g., neo-LVOT, cross-sectional surface area according to certain embodiments. It should be noted that in certain embodiments, process 1700 is a computer-implemented process. Further, certain blocks may be performed automatically, manually by a user of a computing device, or partially manually and partially automatically such as based on input from a user of a computing device. The user may be a medical professional or a non-medical professional, such as a technician or an engineer. Further, certain blocks may be optional, and parts of the described method may be performed as separate methods. More detailed descriptions of some of the process steps of process 1700 may be found above.

Process 1700 begins at block 1702, by generating a model of a structure based on a plurality of images of the structure, the structure comprising at least one fluid flow path. As described above, the model may be a 2D image, a set of 2D images or a virtual 3D or 4D model. In some examples, a model created earlier may be loaded from a file, a database or any other suitable medium. In some examples, block 1702 may comprise acquiring images, e.g., by using scanning device 322 and/or by retrieving them from image data storage 306. In some examples, a virtual 3D or 4D model may be created based on a plurality of images, e.g., by image processing module 308. In certain examples, the structure may comprise (a part of) a patient's heart, a left ventricle, an LVOT, a neo-LVOT, a mitral valve and/or an aortic valve.

The process 1700 may proceed to block 1704 by identifying an obstruction element in the model of the structure, the obstruction element affecting the at least one fluid flow path in the model. Identifying the obstruction element may comprise localizing a present, e.g., pathological, phenomenon, such as a stenosis, blood clot, tumor or calcification. Additionally or alternatively, identifying the obstruction element may comprise localizing a present medically introduced obstruction, such as a medical, e.g., prosthetic, device, and/or creating a virtual treatment plan for the introduction of such a medically introduced obstruction, e.g., by means of virtually introducing a model of the obstruction (e.g., a virtual model of a prosthetic device) into the model of the structure to create an adapted model of the structure. In some examples, block 1704 may be performed by means of image processing module 308 and/or 3D measurement and analysis module 320. In some examples, the obstruction element may comprise a prosthetic mitral valve.

The process 1700 may proceed to block 1706 by determining a region of the at least one fluid flow path for flow analysis. In some examples, the region may be determined as described above. In some examples, the region may comprise one or more of the LVOT, the neo-LVOT and an extension of the neo-LVOT. In some examples, the flow analysis includes the processes shown in blocks 1708 through 1714. For example, process 1700 may proceed to block 1708 by defining a guide curve along at least a portion of the at least one fluid flow path, as described above. The process 1700 may proceed to block 1710 by defining a plurality of planes along the guide curve, each of the plurality of planes orthogonal to the guide curve and intersecting the region to define at least some of a plurality of cross sections of the region, as described above. The process 1700 may proceed to block 1712 by determining which one of the plurality of cross sections has a smallest surface area, as described above. The process 1700 may proceed to block 1714 by displaying a size of the smallest surface area.

In some examples, the obstruction element is a virtual model of a prosthetic device inserted into the model.

In some examples, the process 1700 further includes selecting the virtual model of the obstruction element based on a treatment plan to improve fluid flow in a second fluid flow path.

In some examples, defining the guide curve includes selecting a first point at a first location of the at least one fluid flow path; and selecting a second point at a second location of the at least one fluid flow path, wherein defining the guide curve comprises defining the guide curve from the first point to the second point such that the guide curve follows a direction of fluid flow through the at least one fluid flow path.

In some examples, defining the guide curve includes determining a centerline of at least a portion of the at least one fluid flow path. The centerline may be determined based on the lumen of the at least one fluid flow path. For example, defining the guide curve may comprise calculating a centerline path of the at least one fluid flow path, wherein the centerline path is bounded by the structure and optionally the obstruction element, and wherein the guide curve follows the centerline path.

In some examples, the process 1700 further includes (a) spacing each of the plurality of planes from another one of the plurality of planes at regular intervals along the guide curve; rotating one or more of the plurality of planes about a point of intersection between the one or more of the plurality of planes and the guide curve, wherein the one or more of the plurality of planes are rotated by an angular increment along a first axis; and determining a surface area of each cross section corresponding to each angular increment along the first axis.

In some examples, the process 1700 further includes (b) rotating one or more of the plurality of planes about a point of intersection between the one or more of the plurality of planes and the guide curve, wherein the one or more of the plurality of planes are rotated by an angular increment along a second axis; and determining a surface area of each cross section corresponding to each angular increment along the second axis.

In some examples, the process 1700 further includes (c) defining a second plurality of planes, each of the second plurality of planes corresponding to a rotation of one of the one or more of the plurality of planes about a first axis perpendicular to the guide curve, each of the second plurality of planes intersecting the region to define at least some of the plurality of cross sections of the region.

In some examples, the process 1700 further includes (d) defining a third plurality of planes, each of the third plurality of planes corresponding to a rotation of one of the one or more of the plurality of planes about a second axis perpendicular to the guide curve and the first axis, each of the second plurality of planes intersecting the region to define at least some of the plurality of cross sections of the region.

In some examples, the process 1700 further includes (e) defining a fourth plurality of planes, each of the fourth plurality of planes corresponding to a rotation of one of the one or more of the plurality of planes, the second plurality of planes or the third plurality of planes about a third axis tangential to the guide curve, each of the second plurality of planes intersecting the region to define at least some of the plurality of cross sections of the region.

In some examples, process 1700 includes steps (a) and (b) as described above. In some examples, process 1700 includes steps (a), (c) and (d) as described above. In some examples, process 1700 includes steps (a), (c) and (e) as described above.

In some examples, the process 1700 further includes dividing the plurality of planes into two or more subsets, wherein each of the two or more subsets include one or more planes, and wherein determining which one of the plurality of cross sections has the smallest surface area comprises determining which one of the one or more planes in each subset has the smallest surface area.

In some examples, each subset consists of a selection of planes sharing an intersection point with the guide curve.

In some examples, process 1700 further includes, based on the smallest surface area of each of the two or more subsets, profiling the smallest surface area along the guide curve to generate a smallest surface area profile.

In some examples, the process 1700 further includes, based on the smallest surface area profile, identifying a discrete stenosis or a tunnel stenosis. In some examples, identifying a discrete stenosis or a tunnel stenosis may be done as described above, e.g., by comparing the length of a narrowing with a pre-set threshold. To this end, process 1700 may further include the steps of: defining a first threshold for the cross-sectional surface area; defining a second threshold for the stenosis type; identifying on the smallest surface area profile a sequence of data points below the first threshold; comparing the length of the sequence with the second threshold; and identifying a discrete stenosis if the length of the sequence is below the second threshold and a tunnel stenosis if the length of the sequence is above the second threshold. The first threshold may be an absolute (e.g., expressed as a surface area) or a relative threshold (e.g., expressed as a ratio between a cross-sectional surface area of the adapted model and a corresponding cross-sectional surface area of the original model, or as a ratio between a cross-sectional surface area at a certain point along the profile and the average cross-sectional surface area along the entire profile or of surrounding sections of the profile. The second threshold may be expressed, e.g., in terms of length along the guide curve or in terms of number of datapoints along the profile.

In some examples, the plurality of images comprises a plurality of computed tomography (CT) scans of the structure.

In some examples, determining the region of the at least one fluid flow path further includes utilizing feature-recognition software to identify a portion of the structure that contains the region.

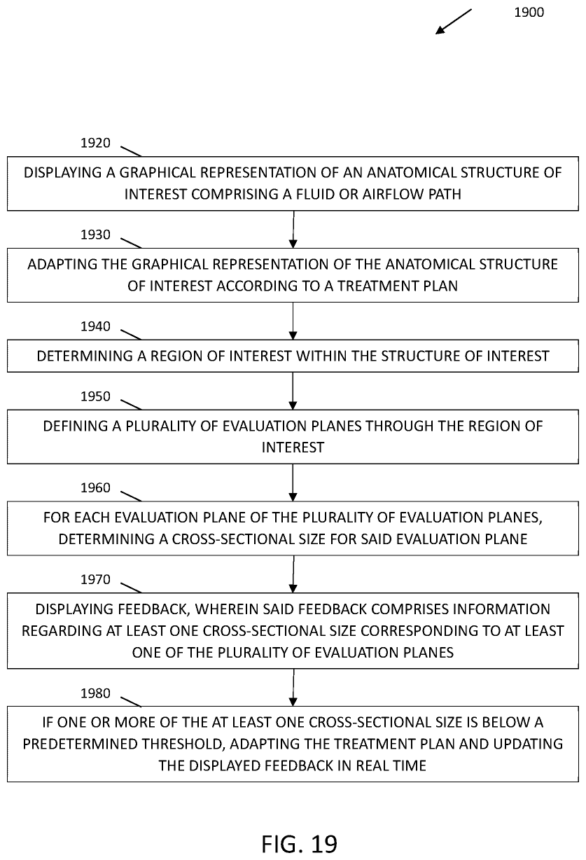

FIG. 19 is a flow chart illustrating a process 1900 for creating a treatment plan for a medical treatment of an anatomical structure of interest of a patient according to certain embodiments. It should be noted that in certain embodiments, process 1900 is a computer-implemented process. Further, certain blocks may be performed automatically, manually by a user of a computing device, e.g., computing device 400, or partially manually and partially automatically such as based on input from a user of a computing device. The user may be a medical professional or a non-medical professional, such as a technician or an engineer. Further, certain blocks may be optional, and parts of the described method may be performed as separate methods. More detailed descriptions of some of the process steps of process 1900 may be found above.

Process 1900 begins at block 1920, by displaying a graphical representation 602, 1822 of at least part of the structure of interest, wherein the structure of interest comprises a fluid or airflow path. As described above, the graphical representation may be a 2D image, a set of 2D images, a virtual 3D model, such as a surface model, or a virtual 4D model. In some examples, a model created earlier may be loaded from a file, a database or any other suitable medium. In some examples, this block may comprise acquiring one or more images of at least part of the structure of interest, wherein the images contain visual information of the passageway. In some examples, acquiring said one or more images may comprise acquiring the images using a scanning device 322. Additionally or alternatively, acquiring said images may comprise loading the images from an image data storage 306. In some examples, the images are medical images, such as CT scans, MRI scans, ultrasound images, etc. In some examples, the images may be contrast-enhanced images. In some examples, a virtual 3D or 4D model may be created based on a plurality of images, e.g., by image processing module 308. In certain examples, the structure may comprise (a part of) a patient's heart, a left ventricle, an LVOT, a neo-LVOT, a mitral valve and/or an aortic valve. The graphical representation may be displayed on a display 404 of computing device 400. The graphical representation may be displayed in one or more image-display areas 1810 and/or 3D-display areas 1820 of a user interface 1800.

Process 1900 may proceed to block 1930 by adapting the graphical representation 602, 1822 of the anatomical structure of interest according to a treatment plan. This may comprise inserting in the graphical representation a virtual model of a prosthetic device 804. In some examples, the virtual model of the prosthetic device 804 may be shaped based on its brand, type, and size. In some examples, the adaptation of the graphic representation of the structure of interest may be made based on a planned orientation and location of the prosthetic device relative to the structure of interest. The virtual model of the prosthetic device 804 may be a 2D or 3D model of the prosthetic device 804. It is incorporated into one or more 2D medical images and/or a virtual 3D model of the structure of interest. The virtual model of the prosthetic device 804 may include a schematic representation, such as a cylinder, a truncated cone, a spindle, a double truncated cone or a combination of one or more such or other primitive shapes, and/or a more lifelike 3D model of the actual device. Additionally or alternatively, it may be or include a parameterized virtual object, a model loaded from a file, or a model loaded from a library of device templates. It may be or include a model based on a CAD design of the prosthetic device, on a (e.g., optical) scan of a physical device or on a segmentation of one or more (e.g., medical) images comprising a depiction of a prosthetic device. The virtual placement of prosthetic device 804 within the patient's anatomy may correspond to the planned outcome of the treatment. The treatment plan may comprise a selection of a prosthetic device (e.g., one or more of a brand, type and size of a prosthetic device). The treatment plan may further comprise a selection of a position (e.g., location and/or orientation) of the prosthetic device relative to the structure of interest. In some examples, the treatment plan may be created by a user by visually or automatically identifying anatomical structures in the display, and/or based on a planning algorithm dictating prosthetic device 804 selection, location, rotation and/or position. In some examples, the treatment plan may be created automatically, e.g., by computing device 400, based on automatically identified anatomical structures and/or anatomical structures previously identified by a user and/or based on a planning algorithm dictating prosthetic device 804 selection and location. In some examples, the device size selection and/or the selection of the position (e.g., location and/or orientation) of the device is made with respect to the patient's anatomy. For example, with respect to features of the patient's anatomy, to anatomical landmarks, to geometrical primitives, such as coordinate systems, planes, lines, vectors or points, derived from such features or landmarks, or to measurements taken of or among such features, landmarks or geometrical primitives. In some examples, the treatment plan may be loaded from a data storage, such as a file, database or any other suitable medium.

Process 1900 may proceed to block 1940 by determining a region of interest 902 within the structure of interest. Computing device 400 may be used to determine the region of interest 902 using the displayed structure of interest 602, 1822 with prosthetic device 804. In some examples, region of interest 902 may be determined by a user and/or determined by an algorithm. For example, the region of interest 902 may be indicated manually by the user on the graphical representation of the structure of interest 602, 1822 (e.g., prior to incorporation of the prosthetic device as shown in FIG. 7) or on the adapted graphical representation of the structure of interest (e.g., after incorporation of the prosthetic device as shown in FIGS. 8 and 9). In another example, the region of interest may be determined automatically by an algorithm based on, for example, manually or algorithmically identified anatomical structures in the structure of interest 711 and/or surrounding areas. The region of interest 902 may encompass the entire structure of interest 711, or only a part of the structure where obstruction of the fluid or airflow may be expected. In some examples, the region of interest 902 may include one or more of the LVOT 106a, the neo-LVOT 106b and an extension of the neo-LVOT 106c. In some examples, the region of interest 902 may encompass the entire left ventricle, optionally excluding the volume occupied by the 3D virtual model of a prosthetic device 804. In some embodiments, calcifications and/or pre-existing hardware are subtracted from the blood pool volume or part of the blood pool volume to determine the region of interest. In some embodiments, the region of interest and/or its limits may be visualized instead of, in addition to or overlaid onto the adapted depiction.

Process 1900 may proceed to block 1950 by determining a plurality of evaluation planes through the region of interest. In typical examples, this is a fully automatic step performed by the computing device. In other examples, some user input may be required. In some examples, the plurality of evaluation planes may be defined as a plurality of parallel planes at regular intervals and perpendicular to a flow direction in the region of interest, such as near the fluid flow exit. In some examples, a plurality of planes at regular intervals and parallel to an aortic valve is determined. In some examples, intervals between 1 mm and 2 cm (e.g., 2 mm, 3 mm, 4 mm, 5 mm, 6 mm, 7 mm, 8 mm, 9 mm, 1 cm, or 1.5 cm) are used. Alternatively, the plurality of evaluation planes may be determined based on a guide curve. Block 1950 may comprise a step of defining a guide curve 1102 through the region of interest. In some examples, a location and direction of a guide curve 1102 may be determined, and a graphic representation of the guide curve 1102 incorporated into the depiction of the structure of interest 711 (e.g., prior to incorporation of the prosthetic device) or on the adapted depiction of the structure of interest (e.g., after incorporation of the prosthetic device). In some examples, the guide curve 1102 comprises a centerline of the region of interest. In other examples, the guide curve 1102 may be generated between two points selected by the user or an algorithm. For example, the user may arbitrarily select two points on a displayed structure of interest 711. Alternatively, an algorithm may select two points at opposite ends of the structure of interest 711 or region of interest. In some examples, a first point may be: (i) an arbitrary point in the structure of interest 711, (ii) a manually indicated point in the structure of interest 711, (iii) an anatomical landmark of the structure of interest 711 (e.g., the apex of the left ventricle), or (iv) the center of the structure of interest 711 or the region of interest, either including or excluding the volume of the representation of the prosthetic device 804 (e.g., the center of gravity of the blood pool volume of the left ventricle, optionally excluding the volume of the prosthetic device 804 in its planned position, any calcification, and/or any pre-existing hardware). A second point may be a point near the fluid flow exit of the structure of interest 711, such as an arbitrary point near the aortic valve, a manually indicated point near the aortic valve, or an automatically determined point, such as the center of the aortic valve. In some examples, multiple points, such as control points or points on the guide curve, may be added between the first and second points. In some examples, the guide curve may comprise one or more straight-line segments, and/or one or more curved segments, such as spline curves, between the selected points. In some examples, the guide curve is defined such that its direction near the fluid flow exit is substantially parallel to the fluid flow path (e.g., perpendicular to the aortic valve or a best-fit plane through the aortic valve). In some examples, the guide curve is defined as a straight line segment of a fixed length ending near the fluid flow exit and parallel to the flow direction near the fluid flow exit, e.g., a line segment of half the mediolateral width of the left ventricle, perpendicular to the aortic valve, starting within the left ventricle and ending in the geometric center point of the aortic valve. Guide curve 1102 may be visually displayed as an object on the graphical representation 602, 1822 of the structure of interest. After definition of the guide curve, the plurality of evaluation planes may be defined based on the guide curve 1102. In some examples, the plurality of evaluation planes comprises a first plurality of planes orthogonal to the guide curve. In some embodiments, the first plurality of planes are spaced at (e.g., regular) intervals along guide curve 1102. The intervals may be determined by an algorithm based on the length of guide curve 1102, the gradient of the anatomical structure(s) along the guide curve 1102, etc. Smaller intervals may be chosen in zones where the likelihood of an obstruction is higher and vice versa. The planes may be spaced at intervals between 1 mm and 2 cm (e.g., 2 mm, 3 mm, 4 mm, 5 mm, 6 mm, 7 mm, 8 mm, 9 mm, 1 cm, or 1.5 cm). The plurality of evaluation planes may further comprise a second plurality of planes obtained by rotating each of the first plurality of planes one or more times over an angular increment about a first axis perpendicular to the guide curve. The plurality of evaluation planes may further comprise a third plurality of planes obtained by rotating each of the first plurality of planes one or more times over an angular increment about a second axis perpendicular to the guide curve and to the first axis. Additionally or alternatively, the plurality of evaluation planes may further comprise a fourth plurality of planes obtained by rotating each of the second plurality of planes one or more times over an angular increment about a third axis tangential to the guide curve. In some examples, the angular increments are among the group of 1°, 2°, 5°, 10°, 12°, or 15°.

Process 1900 may proceed to block 1960, by determining a cross-sectional size for each evaluation plane of the plurality of evaluation planes. In typical examples, this is a fully automatic step performed by the computing device. In some examples, a surface area of a cross section of the anatomical structure (e.g., in the region of interest) where each of the plurality of evaluation planes intersects with the anatomical structure may be determined as the cross-sectional size. Cross sections may be determined of the native anatomical structure, or the presence of the prosthetic may be taken into account in the determination of cross sections. In some examples, one or more evaluation planes may be discarded based on their position with respect to: (i) the representation of the prosthetic device, (ii) the structure of interest, (iii) the region of interest, or (iv) the boundaries of any of these entities. In some examples, the smallest cross section is determined by finding the smallest surface area among all, or remaining, evaluation planes. In some examples, the plurality of planes defined along the guide curve may be divided into a plurality of subsets of one or more planes that share an intersection point with the guide curve. In this example, a smallest cross section among the one or more planes in a subset is determined for each subset. In some examples, the plurality of evaluation planes is reduced to a single plane per subset corresponding to the smallest cross-sectional size for that subset. In some examples, the cross-sectional size for an evaluation plane is determined by determining the cross section through the original structure of interest along the evaluation plane, determining the cross section through the prosthetic device along the evaluation plane, subtracting the cross section through the prosthetic device from the cross section through the original structure of interest, and calculating the surface area of the resulting contour. In some examples, the determined cross-sectional sizes through the original structure of interest—e.g., without the prosthetic device—are stored for later use.

Process 1900 may proceed to block 1970, by displaying feedback, wherein said feedback comprises information regarding at least one cross-sectional size corresponding to at least one of the plurality of evaluation planes. In some examples, the feedback may comprise information regarding a smallest cross-sectional size through the graphical representation of the original structure of interest (e.g., the LVOT). Additionally or alternatively, the feedback may comprise information regarding a smallest cross-sectional size through the adapted graphical representation of the structure of interest (e.g., the LVOT/neo-LVOT/extension of the neo-LVOT). In some examples, displaying the feedback may comprise displaying the at least one cross-sectional size as a numerical value, e.g., by means of interface elements 1843 of user interface 1800. In some examples, displaying the feedback may comprise displaying the cross section corresponding to the at least one cross-sectional size together with the graphical representation of the structure of interest, e.g., in one or more image-display areas 1810 or 3D-display areas 1820 of user interface 1800. In some examples, displaying said cross section together with the graphical representation of the structure of interest in one or more image-display areas 1810 may comprise generating one or more MPR images such that the slice of at least one MPR image coincides with the plane of the cross section, displaying the one or more MPR images in the one or more image-display areas 1810 and displaying the cross section as an overlay onto said at least one MPR image. In some examples, displaying the feedback may comprise displaying a false-color overlay onto the graphical representation of the anatomical structure of interest 1822 as described above.

Process 1900 may proceed to block 1980, by, if one or more of the at least one cross-sectional size is below a predetermined threshold, adapting the treatment plan and updating the displayed feedback in real time. In some examples, adapting the treatment plan may comprise at least one of selecting a different prosthetic device (e.g., replacing the model of the original prosthetic device with a model of said different prosthetic device in the adapted graphical representation of the structure of interest), and selecting a different position (e.g., location and/or orientation) of the prosthetic device. In some examples, the treatment plan is adapted by the computing device based on input from a user. In some examples, updating the displayed feedback may comprise determining the feedback based on the adapted treatment plan and displaying it to the user. In typical examples, updating the displayed feedback is a fully automatic step performed by the computing device. In some examples, updating the displayed feedback may comprise, for each of the evaluation planes of the plurality of evaluation planes, determining a new cross-sectional size. In some examples, updating the displayed feedback may not comprise defining a new plurality of evaluation planes. In some examples, the updated cross-sectional size for an evaluation plane is determined by retrieving the cross section through the original structure of interest along the evaluation plane determined (and stored) in block 1960, determining the cross section through the prosthetic device according to the adapted treatment plan along the evaluation plane, subtracting the cross section through the prosthetic device from the cross section through the original structure of interest, and calculating the surface area of the resulting contour. In some examples, the cross-sectional size is only updated for those evaluation planes that intersect the prosthetic device, and retrieving the cross section through the original structure of interest along the evaluation plane determined (and stored) in block 1960 for other evaluation planes.

In some examples, process 1900 may comprise further steps. For example, process 1900 may comprise a step of generating a report regarding the (adapted) treatment plan. The report may comprise the selected prosthetic device (e.g., brand, type, size) and its position (e.g., location, orientation) relative to the structure of interest. The report may further comprise a graphical representation of the structure of interest, such as a 2D image, a set of 2D images, a virtual 3D model, such as a surface model, a virtual 4D model, or any combination thereof. The report may be a digital file or a tangible file. The report may be stored in a file, database or any other suitable medium. The report may be used for guidance during execution of the treatment. For example, a printed copy of the report may be available to the physician. Additionally or alternatively, the report may be displayed on a display in the operating room. Additionally or alternatively, the report may be displayed by an augmented reality guidance system. For example, the graphical representation of the structure of interest may be registered onto a medical image or set of medical images captured intra-operatively, such as a transesophageal echocardiography (TEE) image, using any suitable image registration techniques known in the art. A model of the planned prosthetic device in its planned position relative to the structure of interest and/or a graphical representation of the structure of interest may then be shown in overlay onto the intra-operatively captured image(s) for visual guidance. The model of the planned prosthetic device and/or the graphical representation of the structure of interest may be shown as 2D models (e.g., as a cross section through a 3D model), 3D models (e.g., opaque, transparent and/or clipped), 4D models (e.g., opaque, transparent and/or clipped), or any combination thereof.

Using the systems and methods described above, a standardized method provides physicians and researchers the ability to determine a minimal neo-LVOT area for transcatheter mitral valve repair research and development as well as determining the appropriate sizing in the context of patient and procedure planning. Although the particular examples above relate to the mitral valve, a skilled artisan will appreciate that the principles, systems, and methods described above can be readily applied in connection with other types of surgical procedures and other areas of the anatomy. For example, in some embodiments, the valve may be a pulmonary branch valve, the tricuspid valve, etc. In other embodiments, the systems and methods described above may be used in the treatment of pulmonary artery stenosis, other valves, left atrial appendage (LAA) closure, stent grafts for aortic aneurysms, brain aneurysm devices, annular assessment (e.g., min/max area), etc. In certain embodiments, the systems and methods described may be used for airways, the treatment of airway conditions and the placement of artificial devices (e.g., stents, grafts, valves, drug-delivery systems, etc.) in airways, etc.

For example, the table below lists a number of applications to which the present teachings apply, mutatis mutandis. The table lists those anatomical features which, in the context of the present disclosure, are each other's equivalents.

| Transcatheter mitral valve | Transcatheter aortic valve | Transcatheter tricuspid |
|---|---|---|
| replacement (TMVR) | replacement (TAVR) | valve replacement (TTVR) |
| Structure of interest: heart, left | Structure of interest: heart, | Structure of interest: heart, |
| heart or left ventricle | left heart or left ventricle, | right heart, right atrium or |
| | including ascending aorta and | right ventricle, including right |
| | coronary arteries | coronary artery |
| Region of interest: left | Region of interest: openings | Region of interest: right |
| ventricle, LVOT/neo- | of the coronary arteries in the | coronary artery |
| LVOT/extension of the neo- | ascending aorta | |
| LVOT | | |
| Cross-sectional size of the | Cross-sectional size of the | Distance from the prosthetic |
| region of interest | region of interest | device to the right coronary |
| | | artery |
| Guide curve through the left | Central axis of each of the | Guide curve through the right |
| ventricle, LVOT/neo- | coronary arteries near the | coronary artery |
| LVOT/extension of the neo- | opening in the ascending aorta | |
| LVOT | | |
| Plurality of evaluation planes | One evaluation plane at the | Plurality of evaluation points |
| (along the guide curve) | opening of each coronary | along the guide curve |
| | artery in the ascending aorta | |
| Calculating a cross section | Calculating a cross section | Calculating a distance from an |
| and its surface area along an | and its surface area along an | evaluation point to the |
| evaluation plane | evaluation plane | prosthetic device |
| Mitral valve | Aortic valve | Tricuspid valve |
| Mitral valve annulus | Aortic valve annulus | Tricuspid valve annulus |
| Prosthetic mitral valve | Prosthetic aortic | Prosthetic tricuspid valve |

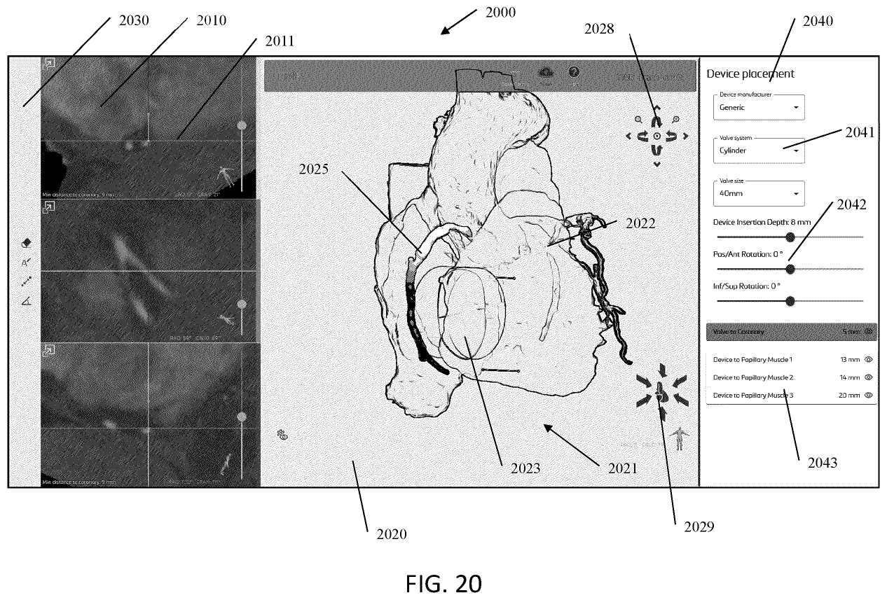

For example, FIG. 20 shows an illustration of a user interface 2000 according to the teachings herein, applied to transcatheter tricuspid valve replacement. In the example shown, user interface 2000 comprises: image-display areas 2010 for displaying 2D images, here with overlaid cross hairs 2011; a 3D-display area 2020, showing an adapted model 2021 of a structure of interest, here a heart 2022, including ascending aorta and coronary arteries; an area 2030 with additional annotation and measurement tools; a treatment-planning area 2040 with user controls 2041 for selecting a prosthetic device, here a prosthetic tricuspid valve to be implanted into the tricuspid valve annulus, and user controls 2042 for determining the position of the prosthetic device relative to the structure of interest; and interface elements 2043 for reporting information regarding the treatment plan to the user. The fluid flow evaluated in this example is blood flow through the region of interest 2025, here the right coronary artery. Placing a too large prosthetic tricuspid valve may cause the right coronary artery to be pinched. The impact of the placement of the prosthetic device on fluid flow in the region of interest is here therefore evaluated by calculating the smallest distance between the planned prosthetic device in its planned position and the region of interest. The smallest distance is reported in interface elements 2043 and is displayed by means of a false-color overlay onto the region of interest 2025. In some embodiments, changing the treatment plan (e.g., selecting a different prosthetic device or changing its planned position with respect to the structure of interest) may cause information displayed in interface elements 2043 and/or the false-color overlay onto structure of interest 2025 to be updated in real time.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims. Further, one or more blocks/steps may be removed or added. For example, only portions of process 1700 illustrated with respect to FIG. 17 may be performed in certain embodiments, such as blocks 1702-1714 to determine which cross section has a smallest surface area.

The methods disclosed herein comprise one or more steps or actions for achieving the described method. Various embodiments disclosed herein provide for the use of a computer system to perform certain or all method steps or actions. A skilled artisan will readily appreciate that such steps may be partly or entirely automated. Various embodiments disclosed herein comprise method steps that are partly or entirely performed by a user, or require the input of a user. A skilled artisan will readily appreciate that this user can be a medical professional, or a non-medical professional, such as a technician or engineer.

Various embodiments disclosed herein refer to computer processing in real time. The term “real time” in the present disclosure should be understood to refer to behavior of a computer system, such as computer environment 300 or computing device 400, in which input data delivered by a user (e.g., a change to the treatment plan) is processed sufficiently fast so that any feedback resulting from the input data (e.g., an updated adapted graphical representation, an updated false-color overlay, an updated MPR image reconstruction, an updated smallest cross section . . . ) is available to the user virtually immediately, e.g., within milliseconds. In the present disclosure, a waiting time of up to 500 milliseconds is regarded to fall within the limits of real-time processing. Various embodiments disclosed herein provide for the use of a computer system to perform certain features. A skilled artisan will readily appreciate that these embodiments may be implemented using numerous different types of computing devices, including both general-purpose and/or special-purpose computing system environments and configurations. Examples of well-known computing systems, environments, and/or configurations that may be suitable for use in connection with the embodiments set forth above may include, but are not limited to, personal computers, server computers, hand-held or laptop devices, multiprocessor systems, microprocessor-based systems, programmable consumer electronics, network PCs, minicomputers, mainframe computers, distributed computing environments that include any of the above systems or devices, and the like. These devices may include stored instructions, which, when executed by a microprocessor in the computing device, cause the computer device to perform specified actions to carry out the instructions. As used herein, instructions refer to computer-implemented steps for processing information in the system. Instructions can be implemented in software, firmware or hardware and include any type of programmed step undertaken by components of the system.

A microprocessor may be any conventional general-purpose single- or multi-chip microprocessor such as a Pentium® processor, a Pentium® Pro processor, a 8051 processor, a MIPS® processor, a Power PC® processor, or an Alpha® processor. In addition, the microprocessor may be any conventional special-purpose microprocessor such as a digital signal processor or a graphics processor. The microprocessor typically has conventional address lines, conventional data lines, and one or more conventional control lines.

Aspects and embodiments disclosed herein may be implemented as a method, apparatus or article of manufacture using standard programming or engineering techniques to produce software, firmware, hardware, or any combination thereof. The term “article of manufacture” as used herein refers to code or logic implemented in hardware or non-transitory computer readable media such as optical storage devices, and volatile or non-volatile memory devices or transitory computer readable media such as signals, carrier waves, etc. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices. These devices may include stored instructions, which, when executed by a microprocessor in the computing device, cause the computer device to perform specified actions to carry out the instructions. As used herein, instructions refer to computer-implemented steps for processing information in the system. Instructions can be implemented in software, firmware or hardware and include any type of programmed step undertaken by components of the system.

Certain aspects of the present disclosure may be summarized in the following clauses.
1. A computer-implemented method for creating a treatment plan for a medical treatment on an anatomical structure of interest of a patient, said anatomical structure of interest comprising a fluid or air passageway defining a fluid or airflow path, said process comprising:
   a) displaying a graphical representation of at least part of the anatomical structure of interest;
   b) adapting the graphical representation of the anatomical structure of interest according to a treatment plan;
   c) determining a region of interest within the anatomical structure of interest;
   d) determining a plurality of evaluation planes through the region of interest;
   e) determining for each evaluation plane of the plurality of evaluation planes a cross-sectional size for said evaluation plane;
   f) displaying feedback regarding the treatment plan, wherein said feedback comprises information regarding at least one cross-sectional size corresponding to at least one of the plurality of evaluation planes; and
   g) if one or more of the at least one cross-sectional size is below a predetermined value, adapting the treatment plan, adapting the graphical representation of the anatomical structure of interest according to the adapted treatment plan and updating the displayed feedback in real time.
2. The method of clause 1, wherein one or more of the steps of adapting the graphical representation of the anatomical structure of interest according to the treatment plan, determining the region of interest within the anatomical structure of interest, determining the plurality of evaluation planes through the region of interest and adapting the treatment plan comprises receiving input from a user, such as a medical professional or a non-medical professional.
3. The method of clause 1 or 2, wherein the anatomical structure of interest comprises one or more of a heart of the patient, a part of the heart, a left ventricle, a left ventricle outflow tract (LVOT), a neo-LVOT, a mitral valve and an aortic valve.
4. The method of any one of clauses 1-3, wherein the graphical representation comprises a 2D image, a set of 2D images, a virtual 3D model, a surface model or a virtual 4D model.
5. The method of any one of clauses 1-4, wherein displaying the graphical representation comprises loading a model from a data storage and displaying said model.
6. The method of any one of clauses 1-5, wherein displaying the graphical representation further comprises acquiring a plurality of images of at least part of the anatomical structure of interest, wherein the images contain visual information of the passageway.
7. The method of clause 6, wherein the plurality of images comprises medical images, CT scans, MRI scans, ultrasound images, or contrast-enhanced images.
8. The method of clause 6 or 7, wherein acquiring the plurality of images comprises acquiring the plurality of images using a scanning device.
9. The method of any one of clauses 6-8, wherein acquiring the plurality of images comprises loading the plurality of images from an image data storage.
10. The method of any one of clauses 6-9, wherein displaying the graphical representation further comprises creating a virtual 3D or 4D model based on the plurality of images.
11. The method of any one of clauses 1-10, wherein displaying the graphical representation of at least part of the anatomical structure of interest comprises displaying the graphical representation on a display of a computing device and/or displaying the graphical representation in one or more image-display areas and/or 3D-display areas of a user interface.
12. The method of any one of clauses 1-11, wherein adapting the graphical representation of the anatomical structure of interest according to the treatment plan comprises inserting a first virtual model of a first prosthetic device in the graphical representation of the anatomical structure of interest in a first location and first orientation according to the treatment plan.
13. The method of clause 12, wherein adapting the treatment plan comprises inserting a second virtual model of a second prosthetic device in the graphical representation of the anatomical structure of interest in a second location and second orientation according to the adapted treatment plan, wherein at least one of:
   the second location is different from the first location,
   the second orientation is different from the first orientation, and
   the second virtual model of the second prosthetic device is different from the first virtual model of the first prosthetic device.
14. The method of clause 12 or 13, wherein the first prosthetic device is a prosthetic mitral valve.
15. The method of any one of clauses 12-14, wherein the virtual model of the prosthetic device comprises one or more of a schematic representation, a cylinder, a truncated cone, a spindle, a double truncated cone, a combination of one or more primitive shapes, a lifelike 3D model, a parameterized virtual object, a model loaded from a file, a model loaded from a library, a model based on a CAD design, and a model based on a scan.
16. The method of any one of clauses 1-15, wherein adapting the graphical representation of the anatomical structure of interest according to a treatment plan comprises creating a treatment plan.
17. The method of any one of clauses 1-16, wherein adapting the graphical representation of the anatomical structure of interest according to a treatment plan comprises loading the treatment plan from a data storage.
18. The method of any one of clauses 1-17, wherein the region of interest encompasses the anatomical structure of interest.
19. The method of any one of clauses 1-17, wherein the region of interest encompasses a part of the anatomical structure of interest, said part comprising the passageway.
20. The method of any one of clauses 1-19, further comprising displaying the region of interest.
21. The method of any one of clauses 1-20, wherein the region of interest comprises one or more of an LVOT, a neo-LVOT, an extension of the neo-LVOT, a left ventricle.
22. The method of clause 21, wherein the region of interest does not include one or more of a volume occupied by a virtual model of a prosthetic device, calcifications and pre-existing hardware.
23. The method of any one of clauses 1-22, wherein determining the plurality of evaluation planes comprises defining a plurality of parallel planes at regular intervals and substantially perpendicular to a flow direction in the region of interest.
24. The method of clause 23, wherein the regular intervals are between 1 mm and 2 cm, more particularly 2 mm, 3 mm, 4 mm, 5 mm, 6 mm, 7 mm, 8 mm, 9 mm, 1 cm, or 1.5 cm.
25. The method of clause 23 or 24, wherein the plurality of parallel planes are parallel to an aortic valve.
26. The method of any one of clauses 1-22, wherein determining the plurality of evaluation planes comprises defining a guide curve through the region of interest and determining the plurality of evaluation planes based on the guide curve.
27. The method of clause 26, wherein updating the displayed feedback does not comprise determining a new guide curve.
28. The method of clause 26 or 27, wherein the guide curve comprises one or more straight-line segments, and/or one or more curved segments.
29. The method of any one of clauses 26-28, wherein the guide curve comprises a centerline of the region of interest.
30. The method of any one of clauses 26-28, wherein the guide curve comprises a curve between a first and a second predetermined point.
31. The method of clause 30, wherein the first predetermined point is one of an arbitrary point in the anatomical structure of interest, a manually indicated point in the anatomical structure of interest, an anatomical landmark of the anatomical structure of interest, or a center of the anatomical structure of interest or the region of interest.
32. The method of clause 31, wherein the anatomical structure of interest comprises a left ventricle or wherein the anatomical landmark is an apex of a left ventricle.
33. The method of any one of clauses 30-32, wherein the second predetermined point is a point near an exit of the fluid or airflow path.
34. The method of clause 33, wherein the exit of the fluid or airflow path is an aortic valve, and the second predetermined point is a center of the aortic valve.
35. The method of any one of clauses 26-34, wherein the guide curve has a direction near an exit of the fluid or airflow path which is substantially parallel to the fluid or airflow path.
36. The method of clause 26 or 28, wherein the guide curve is a straight line segment of a fixed length ending near an exit of the fluid or airflow path and parallel to fluid or airflow path near the exit.
37. The method of clause 36, wherein the guide curve is perpendicular to an aortic valve or a best-fit plane through the aortic valve.
38. The method of any one of clauses 26-37, wherein the plurality of evaluation planes comprises a first plurality of planes orthogonal to the guide curve.
39. The method of clause 38, wherein the planes of the first plurality of planes are spaced according to a likelihood of finding an obstruction.
40. The method of clause 38, wherein the planes of the first plurality of planes are spaced at regular intervals along the guide curve.
41. The method of clause 39, wherein the regular intervals are between 1 mm and 2 cm, more particularly 2 mm, 3 mm, 4 mm, 5 mm, 6 mm, 7 mm, 8 mm, 9 mm, 1 cm, or 1.5 cm.
42. The method of any one of clauses 26-41, wherein the plurality of evaluation planes only comprises planes orthogonal to the guide curve.
43. The method of any one of clauses 38-41, wherein the plurality of evaluation planes further comprises a second plurality of planes obtained by rotating each of the first plurality of planes one or more times over an angular increment about a first axis perpendicular to the guide curve.
44. The method of clause 43, wherein the plurality of evaluation planes further comprises a third plurality of planes obtained by rotating each of the first plurality of planes one or more times over an angular increment about a second axis perpendicular to the guide curve and to the first axis.
45. The method of clause 43 or 44, wherein the plurality of evaluation planes further comprises a fourth plurality of planes obtained by rotating each of the second plurality of planes one or more times over an angular increment about a third axis tangential to the guide curve.
46. The method of any one of clauses 43-45, wherein the angular increment is among the group of 1°, 2°, 5°, 10°, 12°, or 15°.
47. The method of any one of clauses 1-46, wherein updating the displayed feedback in real time does not comprise determining a new plurality of evaluation planes through the region of interest.
48. The method of any one of clauses 1-46, wherein determining a cross-sectional size for an evaluation plane comprises determining a cross section of the anatomical structure of interest along the evaluation plane and a surface area of said cross section.
49. The method of clause 48, wherein determining a cross section of the anatomical structure of interest along the evaluation plane comprises determining a cross section through the graphical representation of the anatomical structure of interest along the evaluation plane.
50. The method of clause 48, wherein determining a cross section of the anatomical structure of interest along the evaluation plane comprises determining a cross section through the adapted graphical representation of the anatomical structure of interest along the evaluation plane.
51. The method of any one of clauses 1-50, further comprising determining a smallest cross-sectional size among the cross-sectional sizes for the plurality of evaluation planes.
52. The method of any one of clauses 1-51, wherein the plurality of evaluation planes is determined based on a guide curve and further comprising:
   dividing the plurality of evaluation planes into a plurality of subsets of one or more planes that share an intersection point with the guide curve; and
   determining for each subset a smallest cross-sectional size among the one or more planes in the subset.
53. The method of clause 52, further comprising reducing the plurality of evaluation planes to a single plane per subset, the single plane corresponding to the smallest cross-sectional size for the subset.
54. The method of any one of clauses 1-53, wherein the feedback comprises information regarding a smallest cross-sectional size through the graphical representation of the anatomical structure of interest.
55. The method of clause 54, wherein the smallest cross-sectional size through the graphical representation of the anatomical structure of interest comprises a smallest cross-sectional size through an LVOT.
56. The method of any one of clauses 1-55, wherein the feedback comprises information regarding a smallest cross-sectional size through the adapted graphical representation of the anatomical structure of interest.
57. The method of clause 56, wherein the smallest cross-sectional size through the adapted graphical representation of the anatomical structure of interest comprises a smallest cross-sectional size through a neo-LVOT.
58. The method of any one of clauses 1-57, wherein displaying the feedback comprises displaying the at least one cross-sectional size as a numerical value.
59. The method of any one of clauses 1-58, wherein displaying the feedback comprises displaying a cross section corresponding to the at least one cross-sectional size together with the graphical representation of the anatomical structure of interest or the adapted graphical representation of the anatomical structure of interest.
60. The method of clause 59, wherein displaying said cross section together with the graphical representation of the anatomical structure of interest or the adapted graphical representation of the anatomical structure of interest comprises generating one or more multi-planar reconstruction (MPR) images such that the slice of at least one MPR image of said one or more MPR images coincides with a plane of the cross section, displaying the one or more MPR images and displaying the cross section as an overlay onto said at least one MPR image.
61. The method of any one of clauses 1-60, wherein displaying the feedback comprises displaying a false-color overlay onto the graphical representation of the anatomical structure of interest or the adapted graphical representation of the anatomical structure of interest.
62. The method of any one of clauses 1-61, wherein updating the displayed feedback does not comprise determining a new region of interest within the anatomical structure of interest.
63. The method of any one of clauses 1-62, wherein updating the displayed feedback comprises determining adapted feedback based on the adapted treatment plan and displaying the adapted feedback.
64. The method of any one of clauses 1-63, wherein updating the displayed feedback comprises, for each of the evaluation planes of the plurality of evaluation planes, determining a new cross-sectional size.
65. The method of any one of clauses 1-64, wherein updating the displayed feedback comprises, for each of the evaluation planes of the plurality of evaluation planes, determining a new cross-sectional size without determining a new plurality of evaluation planes.
66. The method of any one of clauses 1-65, wherein adapting the graphical representation of the anatomical structure of interest according to a treatment plan comprises inserting a first virtual model of a first prosthetic device in the graphical representation of the anatomical structure of interest in a first location and first orientation according to the treatment plan and wherein the cross-sectional size for an evaluation plane is determined by:
   determining a first cross section through the graphical representation of the anatomical structure of interest along the evaluation plane;
   determining a second cross section through the first virtual model of first the prosthetic device along the evaluation plane;
   subtracting the second cross section from the first cross section to determine a first contour; and
   calculating a surface area of the first contour.
67. The method of clause 66,
   wherein adapting the treatment plan comprises inserting a second virtual model of a second prosthetic device in the graphical representation of the anatomical structure of interest in a second location and second orientation according to the adapted treatment plan, wherein at least one of:
      the second location is different from the first location,
      the second orientation if different from the first orientation, and
      the second virtual model of the second prosthetic device is different from the first virtual model of the first prosthetic device; and
   wherein updating the displayed feedback in real time comprises for each of the evaluation planes of the plurality of evaluation planes, determining a new cross-sectional size, wherein the new cross-sectional size is determined by:
      determining a second third section through a virtual model of the second prosthetic device along the evaluation plane;
      subtracting the third cross section from the first cross section to determine a second contour; and
      calculating a surface area of the second contour.
68. The method of clause 66,
   wherein determining the cross-sectional size for an evaluation plane comprises storing the surface area of the first contour.
   wherein adapting the treatment plan comprises inserting a second virtual model of a second prosthetic device in the graphical representation of the anatomical structure of interest in a second location and second orientation according to the adapted treatment plan, wherein at least one of:
      the second location is different from the first location,
      the second orientation if different from the first orientation, and
      the second virtual model of the second prosthetic device is different from the first virtual model of the first prosthetic device;
   wherein updating the displayed feedback in real time comprises for each of the evaluation planes of the plurality of evaluation planes, determining a new cross-sectional size;
   wherein, for evaluation planes intersecting the second virtual model of the second prosthetic device in the second location and the second orientation, the new cross-sectional size is determined by:
      determining a second third section through a virtual model of the second prosthetic device along the evaluation plane;
      subtracting the third cross section from the first cross section to determine a second contour; and
      calculating a surface area of the second contour; and
   wherein, for evaluation planes not intersecting the second virtual model of the second prosthetic device in the second location and the second orientation, the new cross-sectional size is determined by retrieving the surface area of the first contour.
69. The method of clause 67 or 68, wherein updating the displayed feedback in real time comprises for each of the evaluation planes of the plurality of evaluation planes, determining the new cross-sectional size without determining a cross section through the graphical representation of the anatomical structure of interest along the evaluation plane.
70. The method of any one of clauses 1-69, further comprising generating a report regarding the treatment plan or the adapted treatment plan.
71. The method of clause 70, wherein the report comprises a prosthetic device and a position of said prosthetic device relative to the anatomical structure of interest.
72. The method of clause 70 or 71, wherein the report comprises a graphical representation of the anatomical structure of interest, such as one or more of a 2D image, a set of 2D images, a virtual 3D model, a surface model, a virtual 4D model.
73. The method of any one of clauses 70-72, further comprising storing the report and displaying the report for guidance during execution of the medical treatment.
74. The method of clause 73, wherein displaying the report for guidance comprises one or more of printing the report, displaying the report on a display device, and displaying the report on an augmented-reality guidance system.
75. An apparatus for creating a treatment plan for a medical treatment on an anatomical structure of interest of a patient, said anatomical structure of interest comprising a fluid or air passageway defining a fluid or airflow path, said apparatus comprising:
   a memory; and
   a processor communicatively coupled to the memory, the processor and the memory configured to perform the method of any one of clauses 1-74.
76. A non-transitory computer-readable storage medium comprising instructions that, when executed by a processor of an apparatus, cause the apparatus to perform the method of any one of clauses 1-74.

1-53. (canceled)

54. A computer-implemented method for creating a treatment plan for a medical treatment on an anatomical structure of interest of a patient, said anatomical structure of interest comprising a fluid or air passageway defining a fluid or airflow path, said process comprising:
a) displaying a graphical representation of at least part of the anatomical structure of interest;
b) adapting the graphical representation of the anatomical structure of interest according to a treatment plan;
c) determining a region of interest within the anatomical structure of interest;
d) determining a plurality of evaluation planes through the region of interest;
e) determining for each evaluation plane of the plurality of evaluation planes a cross-sectional size for said evaluation plane;
f) displaying feedback regarding the treatment plan, wherein said feedback comprises information regarding at least one cross-sectional size corresponding to at least one of the plurality of evaluation planes; and
g) if one or more of the at least one cross-sectional size is below a predetermined value, adapting the treatment plan, adapting the graphical representation of the anatomical structure of interest according to the adapted treatment plan and updating the displayed feedback in real time;
wherein adapting the graphical representation of the anatomical structure of interest according to the treatment plan comprises inserting a first virtual model of a first prosthetic device in the graphical representation of the anatomical structure of interest in a first location and first orientation according to the treatment plan;
wherein adapting the treatment plan comprises inserting a second virtual model of a second prosthetic device in the graphical representation of the anatomical structure of interest in a second location and second orientation according to the adapted treatment plan, wherein at least one of:
the second location is different from the first location,
the second orientation is different from the first orientation, and
the second virtual model of the second prosthetic device is different from the first virtual model of the first prosthetic device; and
wherein updating the displayed feedback in real time comprises, for each of the evaluation planes of the plurality of evaluation planes, determining a new cross-sectional size without determining a new plurality of evaluation planes.

55. The method of claim 54,
wherein the anatomical structure of interest comprises one or more of a heart of the patient, a part of the heart, a left ventricle, a left ventricle outflow tract (LVOT), a neo-LVOT, a mitral valve and an aortic valve,
wherein the first prosthetic device is a prosthetic mitral valve,
wherein the region of interest comprises one or more of an LVOT, a neo-LVOT, an extension of the neo-LVOT and a left ventricle, and
wherein the region of interest does not include one or more of a volume occupied by a virtual model of a prosthetic device, calcifications and pre-existing hardware.

56. The method of claim 54, wherein the graphical representation comprises a 2D image, a set of 2D images, a virtual 3D model, a surface model or a virtual 4D model.

57. The method of claim 54, wherein displaying the graphical representation further comprises creating a virtual 3D or 4D model based on a plurality of medical images.

58. The method of claim 54, wherein the virtual model of the prosthetic device comprises one or more of a schematic representation, a cylinder, a truncated cone, a spindle, a double truncated cone, a combination of one or more primitive shapes or a parameterized virtual object.

59. The method of claim 54, wherein adapting the graphical representation of the anatomical structure of interest according to a treatment plan comprises one or more of creating a treatment plan and loading the treatment plan from a data storage.

60. The method of claim 54, wherein determining the plurality of evaluation planes comprises defining a plurality of parallel planes at regular intervals and substantially perpendicular to a flow direction in the region of interest.

61. The method of claim 54,
wherein determining the plurality of evaluation planes comprises defining a guide curve through the region of interest and determining the plurality of evaluation planes based on the guide curve; and
wherein updating the displayed feedback in real time does not comprise determining a new guide curve.

62. The method of claim 61,
wherein the guide curve comprises one or more straight-line segments, and/or one or more curved segments;
wherein the guide curve comprises a centerline of the region of interest;
wherein the guide curve comprises a curve between a first and a second predetermined point; or
wherein the guide curve is a straight line segment of a fixed length ending near an exit of the fluid or airflow path and parallel to fluid or airflow path near the exit, such as a straight line segment perpendicular to an aortic valve or a best-fit plane through the aortic valve.

63. The method of claim 62,
wherein the first predetermined point is one of:
an arbitrary point in the anatomical structure of interest, such as an arbitrary point in a left ventricle;
a manually indicated point in the anatomical structure of interest, such as a manually indicated point in a left ventricle;
an anatomical landmark of the anatomical structure of interest, such as an apex of a left ventricle; a center of the anatomical structure of interest, such as a center of a left ventricle; or
a center of the region of interest; and
wherein the second predetermined point is a point near an exit of the fluid or airflow path, such as a center of an aortic valve.

64. The method of claim 61, wherein the plurality of evaluation planes only comprises planes orthogonal to the guide curve.

65. The method of claim 61, wherein the plurality of evaluation planes comprises a first plurality of planes orthogonal to the guide curve and wherein the plurality of evaluation planes further comprises:
a second plurality of planes obtained by rotating each of the first plurality of planes one or more times over an angular increment about a first axis perpendicular to the guide curve;
a second plurality of planes obtained by rotating each of the first plurality of planes one or more times over an angular increment about a first axis perpendicular to the guide curve and a third plurality of planes obtained by rotating each of the first plurality of planes one or more times over an angular increment about a second axis perpendicular to the guide curve and to the first axis; or
a second plurality of planes obtained by rotating each of the first plurality of planes one or more times over an angular increment about a first axis perpendicular to the guide curve and a fourth plurality of planes obtained by rotating each of the second plurality of planes one or more times over an angular increment about a third axis tangential to the guide curve.

66. The method of claim 54,
wherein determining a cross-sectional size for an evaluation plane comprises determining a cross section of the anatomical structure of interest along the evaluation plane and a surface area of said cross section; and
wherein determining a cross section of the anatomical structure of interest along the evaluation plane comprises one or more of:
determining a cross section through the graphical representation of the anatomical structure of interest along the evaluation plane; and
determining a cross section through the adapted graphical representation of the anatomical structure of interest along the evaluation plane.

67. The method of claim 54, wherein the plurality of evaluation planes is determined based on a guide curve and further comprising:
dividing the plurality of evaluation planes into a plurality of subsets of one or more planes that share an intersection point with the guide curve;
determining for each subset a smallest cross-sectional size among the one or more planes in the subset; and
reducing the plurality of evaluation planes to a single plane per subset, the single plane corresponding to the smallest cross-sectional size for the subset.

68. The method of claim 54, wherein the feedback comprises one or more of:
information regarding a smallest cross-sectional size through the graphical representation of the anatomical structure of interest, such as a smallest cross-sectional size through an LVOT; and
information regarding a smallest cross-sectional size through the adapted graphical representation of the anatomical structure of interest, such as a smallest cross-sectional size through a neo-LVOT.

69. The method of claim 54, wherein displaying the feedback comprises one or more of:
displaying the at least one cross-sectional size as a numerical value;
displaying a cross section corresponding to the at least one cross-sectional size together with the graphical representation of the anatomical structure of interest or the adapted graphical representation of the anatomical structure of interest; and
displaying a false-color overlay onto the graphical representation of the anatomical structure of interest or the adapted graphical representation of the anatomical structure of interest.

70. The method of claim 54,
wherein the cross-sectional size for an evaluation plane is determined by:
determining a first cross section through graphical representation of the anatomical structure of interest along the evaluation plane;
determining a second cross section through the first virtual model of the first prosthetic device along the evaluation plane;
subtracting the second cross section from the first cross section to determine a first contour; calculating a surface area of the first contour; and
storing the surface area of the first contour,
wherein updating the displayed feedback in real time comprises for each of the evaluation planes of the plurality of evaluation planes, determining a new cross-sectional size;
wherein, for evaluation planes intersecting the second virtual model of the second prosthetic device in the second location and the second orientation, the new cross-sectional size is determined by:
determining a second third section through a virtual model of the second prosthetic device along the evaluation plane;
subtracting the third cross section from the first cross section to determine a second contour; and
calculating a surface area of the second contour; and
wherein, for evaluation planes not intersecting the second virtual model of the second prosthetic device in the second location and the second orientation, the new cross-sectional size is determined by retrieving the surface area of the first contour.

71. The method of claim 54, further comprising generating a report regarding the treatment plan or the adapted treatment plan.

72. An apparatus for creating a treatment plan for a medical treatment on an anatomical structure of interest of a patient, said anatomical structure of interest comprising a fluid or air passageway defining a fluid or airflow path, said apparatus comprising:
a memory; and
a processor communicatively coupled to the memory, the processor and the memory configured to perform the method of claim 54.

73. A non-transitory computer-readable storage medium comprising instructions that, when executed by a processor of an apparatus, cause the apparatus to perform the method of claim 54.
